(19) United States
(12) Patent Application Publication
(10) Pub. No.: US 2025/0059152 A1
(43) Pub. Date: Feb. 20, 2025

(54) CATALYTIC TETRAHYDROCANNABINOL SYNTHESIS AND PRECURSORS

(71) Applicant: Kare Chemical Technologies Inc., Mississauga (CA)

(72) Inventors: Kamaluddin Abdur-Rashid, Mississauga (CA); Kareem Abdur-Rashid, Mississauga (CA); Wenli Jia, Mississauga (CA)

(21) Appl. No.: 18/717,564

(22) PCT Filed: Dec. 8, 2022
(86) PCT No.: PCT/CA2022/051787

§ 371 Date: Jun. 7, 2024

Related U.S. Application Data
(60) Provisional application No. 63/287,713, filed on Dec. 9, 2021.

(51) Int. Cl. C07D 311/80

(52) U.S. Cl. CPC C07D 311/80

(57) ABSTRACT

The present disclosure provides new tetrahydrocannabinol precursor compounds and processes to prepare tetrahydrocannabinol compounds. The disclosure also relates to the use of catalysts and catalytic processes for the preparation of tetrahydrocannabinol compounds from the tetrahydrocannabinol precursors. Compounds provided are of formula (I) and (II).

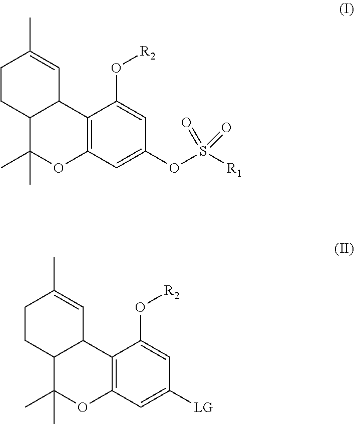

### CROSS-REFERENCE TO RELATED APPLICATIONS

This application claims the benefit of priority to U.S. Provisional Application No. 63/287,713, filed Dec. 9, 2021, the contents of which are incorporated herein by reference in their entirety.

### FIELD OF THE DISCLOSURE

The present disclosure relates to tetrahydrocannabinol precursor compounds and the use of the compounds for the preparation of tetrahydrocannabinol and its analogues. The disclosure also relates to the use of catalysts and catalytic processes for the preparation of tetrahydrocannabinol and its analogues using the tetrahydrocannabinol precursor compounds.

### BACKGROUND OF THE DISCLOSURE

Tetrahydrocannabinol (THC) is the primary psychoactive component and one of the major cannabinoids found in the cannabis plant, from which it can be extracted and purified. THC is used medicinally as an appetite stimulant, antiemetic, and sleep apnea reliever. It is also used to treat anorexia, and chemotherapy-induced nausea and vomiting.

Pure, single component THC is required for pharmaceutical applications. Extracted cannabis resin contains more than 150 cannabinoid compounds, along with terpenes and other compounds present in the plant. Hence, the process of extracting and purifying THC is laborious and time consuming. The yield and quality can also be impacted by environmental factors, weather, drought, pests, pesticides, and residues from the plant and soil.

Commercial extraction is far less feasible for the rare cannabinoid THCV and is essentially impractical for the ultra-rare THC analogues, such as THCB (P. Linciano et al., J. Nat. Prod. 2020, 83, 1, 88-98), THCH (P. Linciano et al., Sci. Rep. 2020, 10, 22019) and THCP (P. Linciano et al., Sci. Rep. 2019, 9, 20335). Some of these compounds are only detectible in miniscule amounts in only a few strains of cannabis.

Several synthetic approaches for THC and its analogues have been described in the prior art. These include the Lewis acid isomerization of CBD (U.S. Pat. No. 5,342,971), asymmetric total synthesis (Carreira et al. J. Am. Chem. Soc. 2017, 139, 18206-18212), and the reaction of menthadienol with olivetol under a variety of reaction conditions (WO 2020051371). Recently, a new process for the catalytic preparation of CBD and CBD analogues, followed by Lewis acid conversions to THC and THC analogues was reported (WO 2020232545).

The prior art reflects the difficulties associated with developing a reliable and commercially viable route for synthetic THC and its analogues.

### SUMMARY OF THE DISCLOSURE

The present disclosure, in some aspects, describes a new approach to the synthesis of tetrahydrocannabinol and its analogues that focuses on the use of novel and stable precursors that can be transformed into the desired tetrahydrocannabinol product on demand. In some embodiments, the novel precursors are derived from commercially available chemicals.

In various aspects, the disclosure relates to the preparation of new tetrahydrocannabinol precursor compounds for the preparation of tetrahydrocannabinol and its analogues and derivatives using catalysts and catalytic processes. In some embodiments, the precursors can be prepared and purified prior to transformation to the desired tetrahydrocannabinol products. The precursors are air-stable and shelf-stable compounds that can be stored, transported, and converted into the desired tetrahydrocannabinol products on demand.

Accordingly, in some embodiments, the present disclosure relates to a tetrahydrocannabinol precursor of Formula (I):

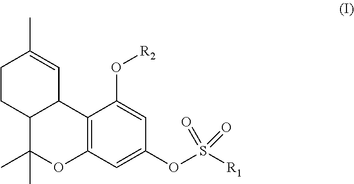

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;
and R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups. In a general way, the compounds of Formula (I) can be prepared and isolated prior to use.

In some other aspects, the compounds and compositions of the disclosure comprise all isomers of compounds of Formula (I). In some other embodiments it provides a mixture of isomers of compounds of Formula (I). In yet some other embodiment it provides single isomers of compounds of Formula (I).

In some other aspects, the present disclosure also relates to tetrahydrocannabinol precursors of Formula (II):

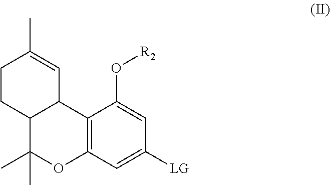

wherein, LG is any suitable leaving group, such as a halo group, sulphonates, or boronates. In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group. In another embodiment, the boronate leaving group is —B(OR)₂, where R is H, a (C₁-C₂₀)-alkyl group (such as a (C₁-C₁₀)-alkyl group) or a (C₆-C₁₄)-aryl group (such as a (C₆-C₁₀)-aryl group). In another embodiment, the boronate leaving group is —BF₃K. In another embodiment, the leaving group is a triflate, mesylate or tosylate group.

In one embodiment, R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups. In a general way, the compounds of Formula (II) can be prepared and isolated prior to use.

In some other aspects, the compounds and compositions of the disclosure comprise all isomers of compounds of Formula (II). In some other embodiments it provides a mixture of isomers of compounds of Formula (II). In yet some other embodiment it provides single isomers of compounds of Formula (II).

In some other aspects, the present disclosure also relates to tetrahydrocannabinol precursors of Formula (I) and Formula (II), wherein one or more of the hydrogen atoms are replaced with deuterium.

In some other aspects, the present disclosure also relates to tetrahydrocannabinol precursors of Formula (I) and Formula (II), wherein one or more of the carbon-12 atoms are replaced with carbon-13.

In various embodiments of the disclosure, the transformations to which the compounds of the disclosure can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the present disclosure to prepare one or more of the tetrahydrocannabinol compounds selected from the group consisting of Formula (III):

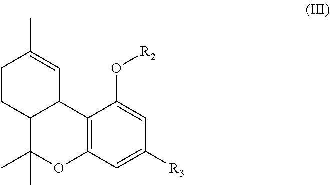

wherein, R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and R₃ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In some other aspects, the compounds and compositions of the disclosure comprise all isomers of compounds of Formula (III). In some other embodiments it provides a mixture of isomers of compounds of Formula (III). In yet some other embodiment it provides single isomers of compounds of Formula (III).

In some other aspects, the present disclosure also relates to the preparation of tetrahydrocannabinol compounds of Formula (III), wherein one or more of the hydrogen atoms are replaced with deuterium.

In some other aspects, the present disclosure also relates to the preparation of tetrahydrocannabinol compounds of Formula (III), wherein one or more of the carbon-12 atoms are replaced with carbon-13 atoms.

In some other aspects of the disclosure, the present disclosure provides a method for the synthesis of one or more of the tetrahydrocannabinol products below:

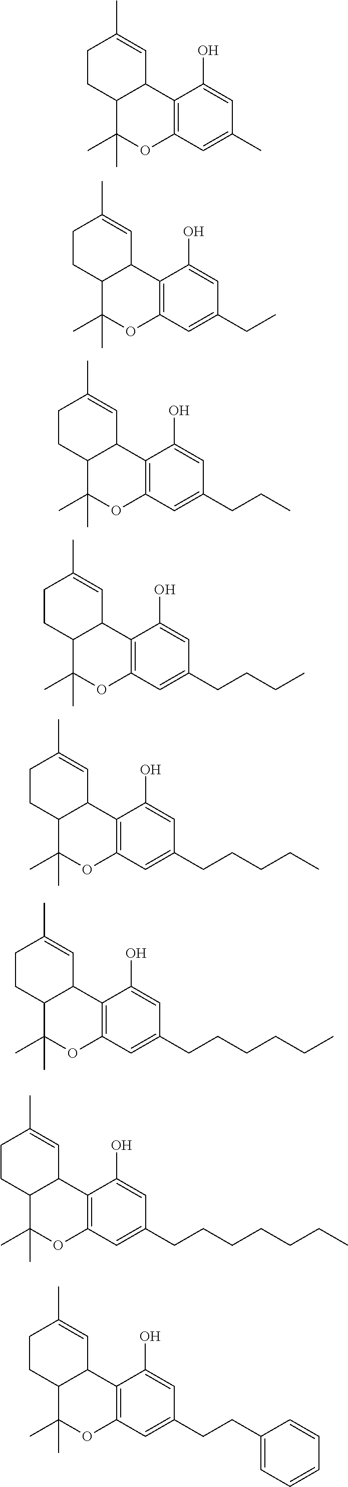

In some other aspects of the disclosure, the present disclosure provides a method for the synthesis of one or more of the deuterated tetrahydrocannabinol products below:

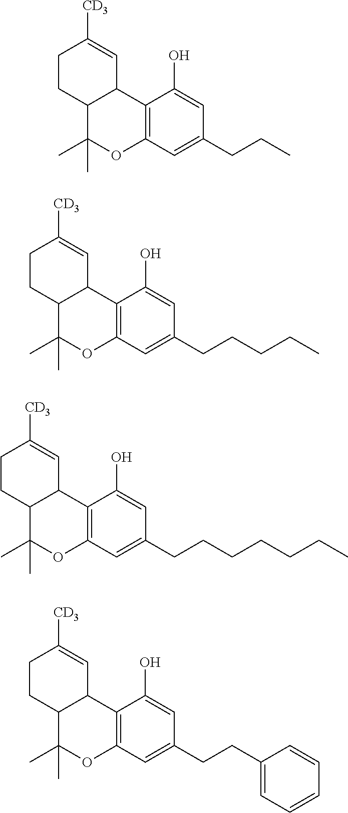

In some other aspects of the disclosure, the present disclosure provides a method for the synthesis of one or more of the deuterated tetrahydrocannabinol products below:

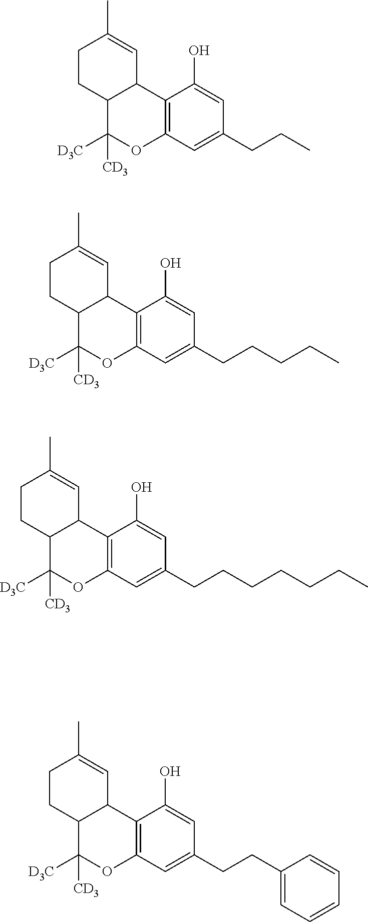 [SMILES: [2H]C([2H])([2H])C1(C([2H])([2H])[2H])Oc2cc(CCC)cc(O)c2C2C=C(C)CCC21 ; [2H]C([2H])([2H])C1(C([2H])([2H])[2H])Oc2cc(CCCCC)cc(O)c2C2C=C(C)CCC21 ; [2H]C([2H])([2H])C1(C([2H])([2H])[2H])Oc2cc(CCCCCCC)cc(O)c2C2C=C(C)CCC21 ; [2H]C([2H])([2H])C1(C([2H])([2H])[2H])Oc2cc(CCc3ccccc3)cc(O)c2C2C=C(C)CCC21]

In some other aspects of the disclosure, the present disclosure provides a method for the synthesis of one or more of the carbon-13 tetrahydrocannabinol products below:

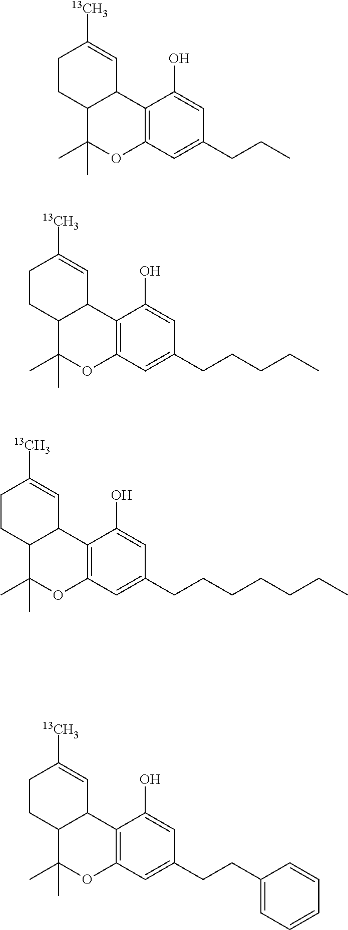 [SMILES: CCCc1cc(O)c2c(c1)OC(C)(C)C1CCC([13CH3])=CC21 ; CCCCCc1cc(O)c2c(c1)OC(C)(C)C1CCC([13CH3])=CC21 ; CCCCCCCc1cc(O)c2c(c1)OC(C)(C)C1CCC([13CH3])=CC21 ; CC1(C)Oc2cc(CCc3ccccc3)cc(O)c2C2C=C([13CH3])CCC21]

In some other aspects of the disclosure, the present disclosure provides a method for the synthesis of one or more of the carbon-13 tetrahydrocannabinol products below:

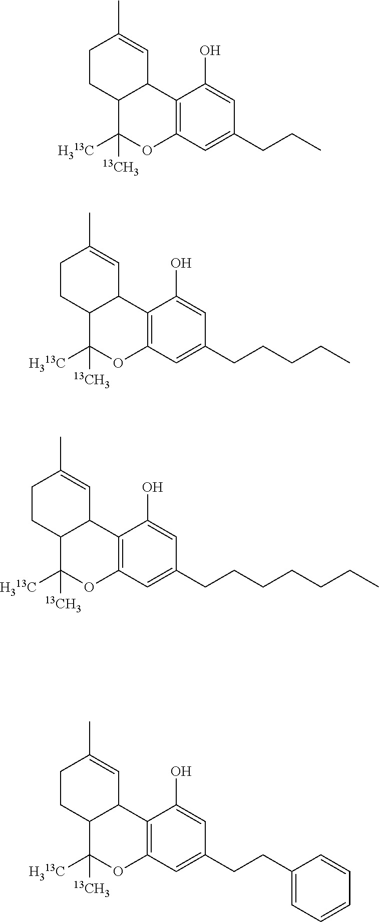 [SMILES: CCCc1cc(O)c2c(c1)OC([13CH3])([13CH3])C1CCC(C)=CC21 ; CCCCCc1cc(O)c2c(c1)OC([13CH3])([13CH3])C1CCC(C)=CC21 ; CCCCCCCc1cc(O)c2c(c1)OC([13CH3])([13CH3])C1CCC(C)=CC21 ; CC1=CC2c3c(O)cc(CCc4ccccc4)cc3OC([13CH3])([13CH3])C2CC1]

In some aspects the disclosure provides a process for the catalytic preparation of compounds of Formula (III).

In some other aspects the disclosure provides a process for the non-catalytic preparation of compounds of Formula (III) from compounds of Formula (I) and Formula (II).

In various embodiments, the process for the preparation of compounds of Formula (III) from compounds of Formula (I) and Formula (II), pursuant to the disclosure uses a boron containing compound such as R₃—B(OH)₂, R₃—B(OR)₂ or R₃—BF₃K.

In some other aspects of the process of the disclosure a Grignard compound such as R₃—MgX is used to prepare compounds of Formula (III).

In still other aspects of the process of the disclosure an organozinc compound such as R₃—ZnX is used to prepare compounds of Formula (III).

The present disclosure also includes, compositions, methods of producing the compounds and compositions comprising the compounds of the disclosure, kits comprising any one or more of the components of the foregoing, optionally with instructions to make or use same and uses of any of the foregoing.

The disclosure also includes the use of compounds of Formula (III), prepared according to the processes of the present disclosure, as pharmaceutical products.

Scheme 1 illustrates the preparation of tetrahydrocannabinol (THC), according to the processes of this disclosure. This is shown as FIG. 1.

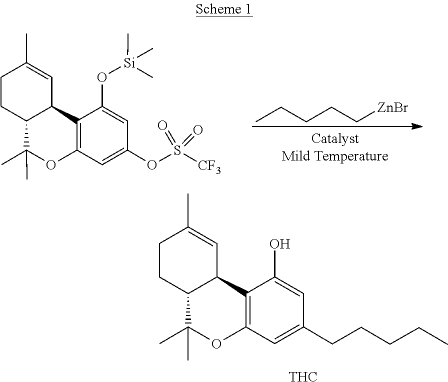

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will be described in greater detail with reference to the following drawings, which are meant to be illustrative by certain embodiments of the disclosure and are not meant to limit the scope of the disclosure:

FIG. 1 shows the scheme for the preparation of tetrahydrocannabinol (THC);

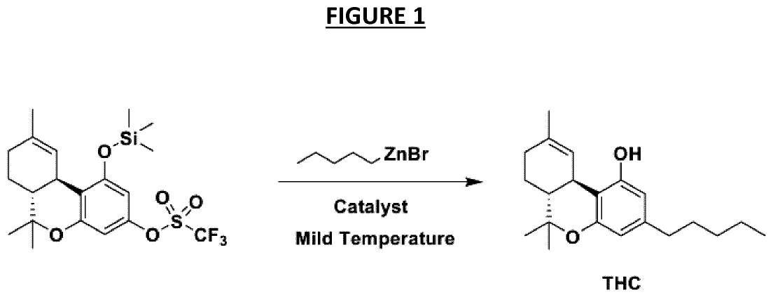

FIG. 2 shows the scheme for the X-ray crystal structure of (6aR,10aR)-6,6,9-trimethyl-3-(trifluoromethylsulfonyloxy)-6a,7,8,10a-tetrahydro-6H-benzo[c]chromen-1-yl benzoate;

FIG. 3 shows the X-ray crystal structure of (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate;

FIG. 4 shows the ¹H NMR spectrum of (6aR,10aR)-1-hydroxy-6,6,9-trimethyl-6a,7,8,10a-tetrahydro-6H-benzo[c]chromen-3-yl trifluoromethanesulfonate;

FIG. 5 shows the ¹H NMR spectrum of (6aR,10aR)-6,6,9-trimethyl-1-(trimethylsilyloxy)-6a,7,8,10a-tetrahydro-6H-benzo[c]chromen-3-yl trifluoromethanesulfonate;

FIG. 6 shows the ¹H NMR spectrum of THC-C1;

FIG. 7 shows the ¹H NMR spectrum of THC-C2;

FIG. 8 shows the ¹H NMR spectrum of THCV;

FIG. 9 shows the ¹H NMR spectrum of THCB;

FIG. 10 shows the ¹H NMR spectrum of THC;

FIG. 11 shows the ¹H NMR spectrum of THCH;

FIG. 12 shows the ¹H NMR spectrum of THCP;

FIG. 13 shows the ¹H NMR spectrum of THC-C8;

FIG. 14 shows the ¹H NMR spectrum of THC-C9;

FIG. 15 shows the ¹H NMR spectrum of THC-C10;

FIG. 16 shows the ¹H NMR spectrum of benzyl-THC;

FIG. 17 shows the ¹H NMR spectrum of (6aR,10aS)-1-hydroxy-6,6,9-trimethyl-6a,7,8,10a-tetrahydro-6H-benzo[c]chromen-3-yl trifluoromethanesulfonate/0.5 Et₂O;

FIG. 18 shows the ¹H NMR spectrum of (6aR,10aS)-6,6,9-trimethyl-1-(trimethylsilyloxy)-6a,7,8,10a-tetrahydro-6H-benzo[c]chromen-3-yl trifluoromethanesulfonate;

FIG. 19 shows the ¹H NMR spectrum of (R,S)-THCV;

FIG. 20 shows the ¹H NMR spectrum of (R,S)-THC;

FIG. 21 shows the ¹H NMR spectrum of (+)-Perrottetinene;

FIG. 22 shows the ¹H NMR spectrum of (6aR,10aR)-6,6,9-trimethyl-3-(trifluoromethylsulfonyloxy)-6a,7,8,10a-tetrahydro-6H-benzo[c]chromen-1-yl benzoate;

FIG. 23 shows the ¹H NMR spectrum of (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate;

FIG. 24 shows the ¹H NMR spectrum of (6aR,10aR)-6,6,9-trimethyl-3-(trifluoromethylsulfonyloxy)-6a,7,8,10a-tetrahydro-6H-benzo[c]chromen-1-yl 2-naphthoate;

FIG. 25 shows the ¹H NMR spectrum of (6aR,10aR)-6,6,9-trimethyl-3-pentyl-6a,7,8,10a-tetrahydro-6H-benzo[c]chromen-1-yl 2-naphthoate.

### DETAILED DESCRIPTION OF THE DISCLOSURE

### (I) Definitions

The term “alkyl” as used herein means straight and/or branched chain, saturated alkyl radicals containing one or more carbon atoms and includes (depending on the identity) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like.

The term “alkenyl” as used herein means straight and/or branched chain, unsaturated alkyl radicals containing two or more carbon atoms and one to three double bonds, and includes (depending on the identity) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like.

The term “alkynyl” as used herein means straight and/or branched chain, unsaturated alkyl radicals containing two or more carbon atoms and one to three triple bonds, and includes (depending on the identity) acetylynyl, propynyl, but-1-ynyl, but-2-ynyl, but-3-ynyl, 3-methylbut-1-enyl, 3-methylpent-1-ynyl, 4-methylpent-1-ynyl, 4-methylpent-2-ynyl, penta-1,3-di-ynyl, hexyn-1-yl and the like.

The term “alkoxy” as used herein means straight and/or branched chain alkoxy group containing one or more carbon atoms and includes (depending on the identity) methoxy, ethoxy, propyloxy, isopropyloxy, t-butoxy, heptoxy, and the like.

The term “cycloalkyl” as used herein means a monocyclic, bicyclic or tricyclic saturated carbocylic group containing three or more carbon atoms and includes (depending on the identity) cyclopropyl, cyclobutyl, cyclopentyl, cyclodecyl and the like.

The term “aryl” as used herein means a monocyclic, bicyclic or tricyclic aromatic ring system containing at least one aromatic ring and 6 or more carbon atoms and includes phenyl, naphthyl, anthracenyl, 1,2-dihydronaphthyl, 1,2,3,4-tetrahydronaphthyl, fluorenyl, indanyl, indenyl and the like.

The term “heteroaryl” as used herein means a monocyclic, bicyclic or tricyclic ring system containing one or two aromatic rings and 5 or more atoms of which, unless otherwise specified, one, two, three, four or five are heteromoieties independently selected from N, NH, N(alkyl), O and S and includes thienyl, furyl, pyrrolyl, pyrididyl, indolyl, quinolyl, isoquinolyl, tetrahydroquinolyl, benzofuryl, benzothienyl and the like.

The term “halo” or “halogen” as used herein means chloro, fluoro, bromo or iodo. The term “fluoro-substituted” as used herein means that at least one, including all, of the hydrogens on the referenced group is replaced with fluorine.

The suffix “ene” added on to any of the above groups means that the group is divalent, i.e. inserted between two other groups.

The term “ring system” as used herein refers to a carbon-containing ring system, that includes monocycles, fused bicyclic and polycyclic rings, bridged rings and metalocenes. Where specified, the carbons in the rings may be substituted or replaced with heteroatoms.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. For instance, “including” also encompasses “including but not limited to”. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

The present disclosure relates to a tetrahydrocannabinol precursor of Formula (I):

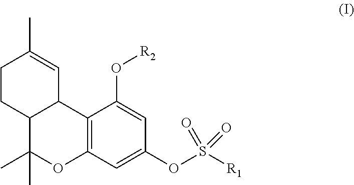

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;
and R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups. In a general way, the compounds of Formula (I) can be prepared and isolated prior to use.

In one embodiment, R₁ represents a hydrogen atom, an optionally substituted (C₁-C₂₀)-alkyl group, an optionally substituted (C₂-C₂₀)-alkenyl group, an optionally substituted (C₂-C₂₀)-alkynyl group, an optionally substituted (C₃-C₂₀)-cycloalkyl group, an optionally substituted (C₆-C₂₀)-aryl group, an optionally substituted (C₅-C₂₀)-heteroaryl group, an optionally substituted OR^{c} group or an optionally substituted NR^{c}₂ group, with possible and non-limiting substituents of R₁ being halogen atoms, OR^{c}, or NR^{c}₂ groups, in which R^{c} is a hydrogen atom, an optionally substituted (C₁-C₂₀)-alkyl group, an optionally substituted (C₂-C₂₀)-alkenyl group, an optionally substituted (C₃-C₂₀)-cycloalkyl group, or an optionally substituted (C₆-C₂₀)-aryl group.

In one embodiment, R₁ represents a hydrogen atom, an optionally substituted (C₁-C₁₀)-alkyl group, an optionally substituted (C₂-C₁₀)-alkenyl group, an optionally substituted (C₂-C₁₀)-alkynyl group, an optionally substituted (C₃-C₂₀)-cycloalkyl group, an optionally substituted (C₆-C₁₀)-aryl group, an optionally substituted (C₅-C₁₀)-heteroaryl group, an optionally substituted OR^{c} group or an optionally substituted NR^{c}₂ group.

In one embodiment, R₁ represents a hydrogen atom, an optionally substituted (C₁-C₆)-alkyl group, an optionally substituted (C₂-C₆)-alkenyl group, an optionally substituted (C₂-C₆)-alkynyl group, an optionally substituted (C₃-C₆)-cycloalkyl group, an optionally substituted (C₆)-aryl group, an optionally substituted (C₅-C₆)-heteroaryl group, an optionally substituted OR^{c} group or an optionally substituted NR^{c}₂ group.

In one embodiment, R₁ represents an optionally substituted (C₁-C₂₀)-alkyl group, in which the one or more optional substituents are a halogen atom, such as fluoro.

In one embodiment, R₁ represents an optionally substituted (C₁-C₁₀)-alkyl group, or (C₁-C₆)-alkyl group, wherein the optional substituents are fluoro. In one embodiment, R₁ represents a fluoro-substituted (C₁-C₂₀)-alkyl group, fluoro-substituted (C₁-C₁₀)-alkyl group or fluoro-substituted (C₁-C₆)-alkyl group. In one embodiment, R₁ is CF₃.

In one embodiment, R₁ represents an optionally substituted (C₆-C₂₀)-aryl group, in which the one or more optional substituents are a halogen atom, such as fluoro, or a (C₁-C₆) alkyl group. In one embodiment, R₁ represents an optionally substituted (C₆-C₁₀)-aryl group, or (C₆)-aryl group, wherein the optional substituents are fluoro or methyl. In one embodiment, R₁ represents a substituted (C₆-C₂₀)-aryl group, a substituted (C₆-C₁₀)-aryl group or a substituted (C₆)-aryl group, in which one or more substituents are F or CH₃.

In one embodiment, R₂ represent hydrogen.

In one embodiment, R₂ represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

In one embodiment, R₂ represent a (C₁-C₁₀)-alkyl group, a (C₂-C₁₀)-alkenyl group, a (C₂-C₁₀)-alkynyl group, a (C₃-C₁₀)-cycloalkyl group, a —Si[(C₁-C₁₀)-alkyl]₃ group, a (C₆-C₁₀)-aryl group, or a (C₅-C₁₀)-heteroaryl group, or an acyl group —C(═O)—R′, wherein R′ is a (C₁-C₁₀)-alkyl group, wherein each group is each optionally substituted with one or more halogen atoms (F, Cl, Br or I), a —(C₁-C₁₀)-alkyl group, a (C₂-C₁₀)-alkenyl group, a (C₂-C₁₀)-alkynyl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl, and
wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₁₀)-alkyl groups.

In one embodiment, R₂ represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms (F, Cl, Br or I), a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and
wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₆)-alkyl groups.

In one embodiment, R₂ represent a (C₁-C₆)-alkyl group, a —Si[(C₁-C₆)-alkyl]₃ group, or a phenyl group.

In one embodiment, R₂ represent a —Si[(C₁-C₆)-alkyl]₃ group. In one embodiment, R₂ represent a —Si[(C₁-C₃)-alkyl]₃ group. In one embodiment, R₂ represent a —Si(CH₃)₃ group.

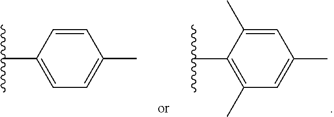

In another embodiment, R₁ represents a hydrogen atom, —CF₃, or.

In one embodiment, the compound of Formula (I) is one of the structures below:

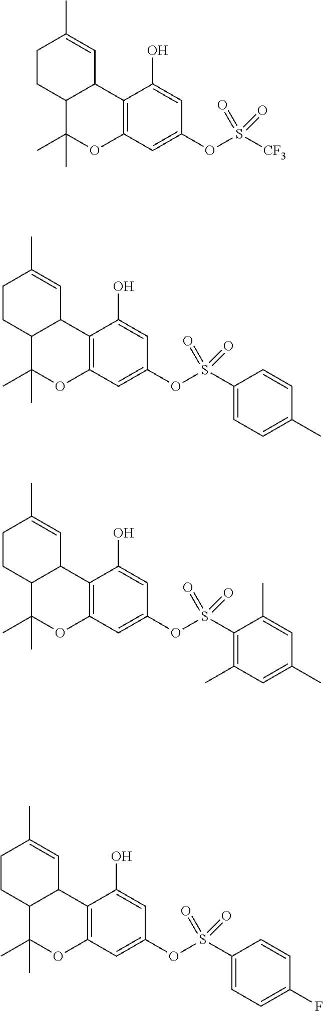 [SMILES: CC1=CC2c3c(O)cc(OS(=O)(=O)C(F)(F)F)cc3OC(C)(C)C2CC1 ; CC1=CC2c3c(O)cc(OS(=O)(=O)c4ccc(C)cc4)cc3OC(C)(C)C2CC1 ; CC1=CC2c3c(O)cc(OS(=O)(=O)c4c(C)cc(C)cc4C)cc3OC(C)(C)C2CC1 ; CC1=CC2c3c(O)cc(OS(=O)(=O)c4ccc(F)cc4)cc3OC(C)(C)C2CC1]

In some other aspects, the compounds and compositions of the disclosure comprise all isomers of compounds of Formula (I). In some other embodiments it provides a mixture of isomers of compounds of Formula (I). In yet some other embodiment it provides single isomers of compounds of Formula (I).

In some other aspects, the compounds and compositions of the disclosure comprise isomers of compounds of Formula (I) shown below:

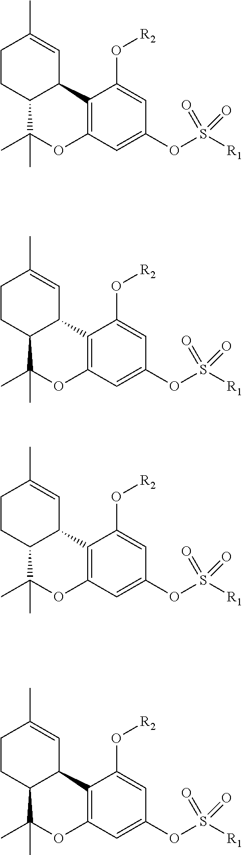

The present disclosure also relates to tetrahydrocannabinol precursors of Formula (II):

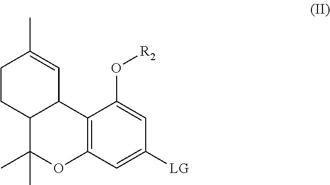

wherein LG is any suitable leaving group. In one embodiment, LG is
   (i) an anionic group such as sulphonates, halides or boronates;
   (ii) MXₙ groups (M=Li, Mg, Zn, Sn, B, Si; X is halide, OH, OR, (C₁-C₂₀)-alkyl, (C₁-C₂₀)-aryl, etc.; n=0 to 3).

In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group. In another embodiment, the boronate leaving group is —B(OR)₂, where R is H, a (C₁-C₂₀)-alkyl group (such as a (C₁-C₁₀)-alkyl group) or a (C₆-C₁₄)-aryl group (such as a (C₆-C₁₀)-aryl group). In another embodiment, the boronate leaving group is —BF₃K.

In another embodiment, the present disclosure relates to tetrahydrocannabinol precursors of Formula (II), wherein, one or more of the hydrogen atoms are replaced with deuterium;
and/or one or more of the carbon-12 atoms are replaced with carbon-13;
and R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

In some other aspects, the compounds and compositions of the disclosure comprise all isomers of compounds of Formula (II). In some other embodiments it provides a mixture of isomers of compounds of Formula (II). In yet some other embodiment it provides single isomers of compounds of Formula (II).

In some other aspects, the compounds and compositions of the disclosure comprise isomers of compounds of Formula (II) shown below:

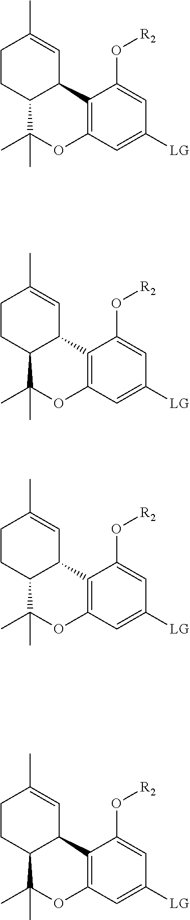

The transformations to which the compounds of the disclosure can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the disclosure to prepare tetrahydrocannabinol compounds of Formula (III):

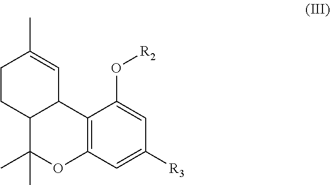

wherein, R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and R₃ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₃ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In one embodiment, R₃ represents a hydrogen atom, a (C₁-C₁₀)-alkyl group, a (C₂-C₁₀)-alkenyl group, a (C₂-C₁₀)-alkynyl group, a (C₃-C₁₀)-cycloalkyl group, a (C₆—C₁₀)-aryl group, wherein the latter 5 groups are each optionally substituted with one or more halogen atoms (F, Cl, Br or I), —(C₁-C₁₀)-alkyl, a (C₂-C₁₀)-alkenyl group, a (C₂-C₁₀)-alkynyl group, (C₆-C₁₀)-aryl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl.

In one embodiment, R₃ represents a hydrogen atom, a (C₁-C₆)-alkyl group, a (C₂-C₆)-alkenyl group, a (C₂-C₆)-alkynyl group, a (C₃-C₆)-cycloalkyl group, a (C₆)-aryl group, wherein the latter 5 groups are each optionally substituted with one or more halogen atoms (F, Cl, Br or I), —(C₁-C₆)-alkyl, a (C₂-C₆)-alkenyl group, a (C₂-C₆)-alkynyl group, (C₆)-aryl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl.

In one embodiment, R₃ represents a hydrogen atom, a (C₁-C₂₀)-alkyl group, a (C₂-C₂₀)-alkenyl group, a (C₆-C₁₄)-aryl group, wherein the latter 3 groups are each optionally substituted with one or more halogen atoms (F, Cl, Br or I), —(C₁-C₁₁)-alkyl, a (C₂-C₁₀)-alkenyl group, a (C₂-C₁₀)-alkynyl group, or (C₆-C₁₀)-aryl group.

In one embodiment, R₃ represents a hydrogen atom, a (C₁-C₆)-alkyl group, a (C₂-C₆)-alkenyl group, a (C₆)-aryl group, wherein the latter 3 groups are each optionally substituted with one or more halogen atoms (F, Cl, Br or I), —(C₁-C₆)-alkyl, a (C₂-C₆)-alkenyl group, a (C₂-C₆)-alkynyl group, or (C₆)-aryl group.

In one embodiment, R₃ represents a hydrogen atom, a (C₁-C₂₀)-alkyl group, a (C₆-C₁₀)-aryl group, wherein the latter 2 groups are each optionally substituted with one or more phenyl groups.

In one embodiment, R₃ represents a hydrogen atom, a (C₁-C₁₀)-alkyl group, a (C₆-C₁₀)-aryl group, wherein the latter 2 groups are each optionally substituted with one or more phenyl groups.

In one embodiment, R₃ represents a hydrogen atom, a (C₁-C₆)-alkyl group, a (C₆)-aryl group, wherein the latter 2 groups are each optionally substituted with one or more phenyl groups.

In one embodiment, R₃ represents a hydrogen atom or a (C₁-C₂₀)-alkyl group optionally substituted with a phenyl group.

In one embodiment, R₃ represents a hydrogen atom or a (C₁-C₁₀)-alkyl group optionally substituted with a phenyl group.

In one embodiment, R₃ represents a hydrogen atom or a (C₁-C₆)-alkyl group optionally substituted with a phenyl group.

In one embodiment, R₂ is as defined in any of the above paragraphs.

In some other aspects, the present disclosure also relates to the preparation of tetrahydrocannabinol compounds of Formula (III), wherein one or more of the hydrogen atoms are replaced with deuterium.

In some other aspects, the present disclosure also relates to the preparation of tetrahydrocannabinol compounds of Formula (III), wherein one or more of the carbon-12 atoms are replaced with carbon-13 atoms.

In some other aspects, the compounds and compositions of the disclosure comprise all isomers of compounds of Formula (III). In some other embodiments it provides a mixture of isomers of compounds of Formula (III). In yet some other embodiment it provides single isomers of compounds of Formula (III).

In some other aspects, the compounds and compositions of the disclosure comprise isomers of compounds of Formula (III) shown below:

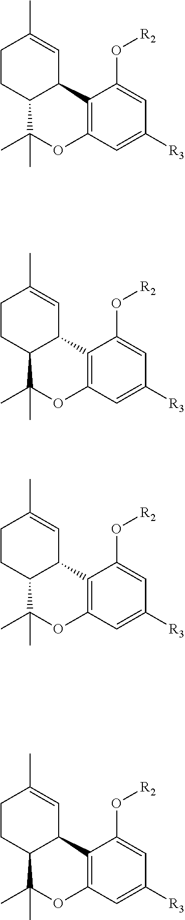

The disclosure also includes the use of compounds of Formula (III), prepared according to the processes of the present disclosure, as pharmaceutical products.

### (III) Processes of the Disclosure

The present disclosure also relates to a process for the production of compounds of Formula (I) comprising contacting a compound of Formula (IV)

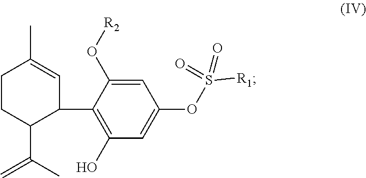

with a catalyst.

In one embodiment, suitable catalysts include but are not limited to Lewis acid catalysts, protic acid catalysts, transition metal salts, transition metal complexes and organocatalysts.

The disclosure also relates to a process for the catalytic and non-catalytic use of compounds of Formula (I) and Formula (II) to prepare tetrahydrocannabinol compounds of Formula (III):

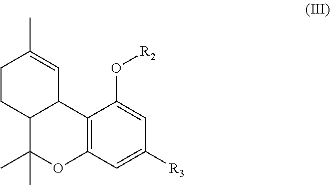

wherein, R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and R₃ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₃ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In one embodiment, R₃ represents a hydrogen atom, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, wherein the latter 5 groups are each optionally substituted with one or more halogen atoms (F, Cl, Br or I), —(C₁-C₁₀)-alkyl, a (C₂-C₁₀)-alkenyl group, a (C₂-C₁₀)-alkynyl group, (C₆-C₁₀)-aryl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl.

In one embodiment, R₃ represents a hydrogen atom, a (C₁-C₆)-alkyl group, a (C₂-C₆)-alkenyl group, a (C₂-C₆)-alkynyl group, a (C₃-C₆)-cycloalkyl group, a (C₆)-aryl group, wherein the latter 5 groups are each optionally substituted with one or more halogen atoms (F, Cl, Br or I), —(C₁-C₆)-alkyl, a (C₂-C₆)-alkenyl group, a (C₂-C₆)-alkynyl group, (C₆)-aryl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl.

In one embodiment, R₃ represents a hydrogen atom, a (C₁-C₂₀)-alkyl group, a (C₂-C₂₀)-alkenyl group, a (C₆-C₁₄)-aryl group, wherein the latter 3 groups are each optionally substituted with one or more halogen atoms (F, Cl, Br or I), —(C₁-C₁₁)-alkyl, a (C₂-C₁₀)-alkenyl group, a (C₂-C₁₀)-alkynyl group, or (C₆-C₁₀)-aryl group.

In one embodiment, R₃ represents a hydrogen atom, a (C₁-C₆)-alkyl group, a (C₂-C₆)-alkenyl group, a (C₆)-aryl group, wherein the latter 3 groups are each optionally substituted with one or more halogen atoms (F, Cl, Br or I), —(C₁-C₆)-alkyl, a (C₂-C₆)-alkenyl group, a (C₂-C₆)-alkynyl group, or (C₆)-aryl group.

In one embodiment, R₃ represents a hydrogen atom, a (C₁-C₂₀)-alkyl group, a (C₆-C₁₀)-aryl group, wherein the latter 2 groups are each optionally substituted with one or more phenyl groups.

In one embodiment, R₃ represents a hydrogen atom, a (C₁-C₁₀)-alkyl group, a (C₆-C₁₀)-aryl group, wherein the latter 2 groups are each optionally substituted with one or more phenyl groups.

In one embodiment, R₃ represents a hydrogen atom, a (C₁-C₆)-alkyl group, a (C₆)-aryl group, wherein the latter 2 groups are each optionally substituted with one or more phenyl groups.

In one embodiment, R₃ represents a hydrogen atom or a (C₁-C₂₀)-alkyl group optionally substituted with a phenyl group.

In one embodiment, R₃ represents a hydrogen atom or a (C₁-C₁₀)-alkyl group optionally substituted with a phenyl group.

In one embodiment, R₃ represents a hydrogen atom or a (C₁-C₆)-alkyl group optionally substituted with a phenyl group.

In some other aspects, the present disclosure also relates to the preparation of tetrahydrocannabinol compounds of Formula (III), wherein one or more of the hydrogen atoms are replaced with deuterium.

In some other aspects, the present disclosure also relates to the preparation of tetrahydrocannabinol compounds of Formula (III), wherein one or more of the carbon-12 atoms are replaced with carbon-13 atoms.

In some other aspects, the compounds and compositions of the disclosure comprise all isomers of compounds of Formula (III). In some other embodiments it provides a mixture of isomers of compounds of Formula (III). In yet some other embodiment it provides single isomers of compounds of Formula (III).

Carbon-carbon bond forming reactions for the preparation of tetrahydrocannabinol compounds of Formula (III) include but are not limited to catalytic and non-catalytic Ullman, Suzuki-Miyaura, Negishi, Kumada, Sonogashira and Stille reactions.

In some embodiments of the disclosure, a compound of Formula (I) or Formula (II) is contacted with a nucleophilic R₃ group, R₃—W, wherein R₃ is as defined above and is nucleophilic and W is an electrophilic group, such as a boron containing compound, such as R₃—B(OH)₂, R₃—B(OR)₂ or R₃—BF₃K; or a Grignard compound such as R₃—MgX; or an organozinc compound, such as R₃—ZnX, wherein X is halo, in the presence or absence of a catalyst to produce a compound of Formula (III).

In one embodiment, compounds of Formula (II) are prepared as in the following examples:

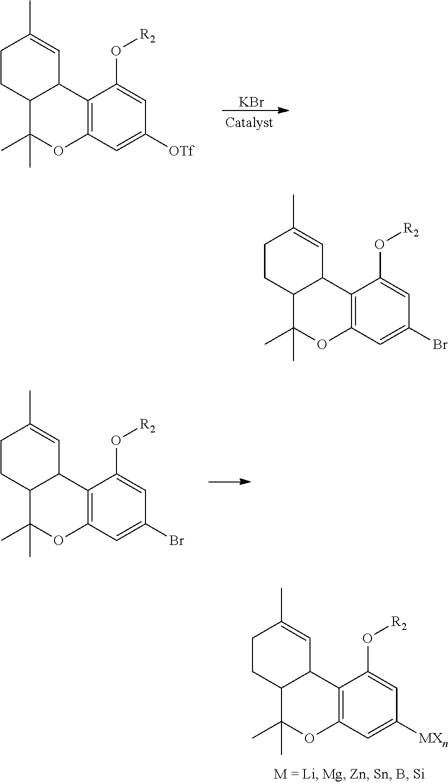

In some embodiments of the disclosure, the catalytic system characterizing the process of the instant disclosure may comprise a base. In some embodiments, said base can be any conventional base. In some embodiments, non-limiting examples include: organic non-coordinating bases such as DBU, an alkaline or alkaline-earth metal carbonate, a carboxylate salt such as sodium or potassium acetate, or an alcoholate or hydroxide salt. Preferred bases are the alcoholate or hydroxide salts selected from the group consisting of the compounds of formula (RO)₂M′ and ROM″, wherein M′ is an alkaline-earth metal, M″ is an alkaline metal and R stands for hydrogen or a linear or branched alkyl group.

The catalyst can be added to the reaction medium in a large range of concentrations. As non-limiting examples, one can cite as catalyst concentration values ranging from 0.001% to 50%, relative to the amount of substrate, thus representing respectively a substrate/catalyst (S/cat) ratio of 100,000 to 2.

Preferably, the complex concentration will be comprised between 0.01% and 10%, i.e. a S/cat ratio of 10,000 to 10 respectively. In some preferred embodiments, there will be used concentrations in the range of 0.1 to 5%, corresponding to a S/cat ratio of 1000 to 20 respectively.

If required, useful quantities of base, added to the reaction mixture, may be comprised in a relatively large range. In some embodiments, non-limiting examples include: ranges between 1 to 100 molar equivalents relative to the substrate. However, it should be noted that it is also possible to add a small amount of base (e.g. base/substrate=1 to 3) to achieve high yields.

In the processes of this disclosure, the catalytic reaction can be carried out in the presence or absence of a solvent. When a solvent is required or used for practical reasons, then any solvent currently used in catalytic reactions can be used for the purposes of the disclosure. Non-limiting examples include aromatic solvents such as benzene, toluene or xylene, hydrocarbon solvents such as hexane or cyclohexane, ethers such as tetrahydrofuran, or yet primary or secondary alcohols, or water, or mixtures thereof. A person skilled in the art is well able to select the solvent most convenient in each case to optimize the catalytic reaction.

The temperature at which the catalytic reaction can be carried out is comprised between −30° C. and 200° C., more preferably in the range of between 0° C. and 100° C. Of course, a person skilled in the art is also able to select the preferred temperature.

Standard catalytic conditions, as used herein, typically implies the mixture of the substrate with the catalyst with or without a base, possibly in the presence of a solvent, and then treating such a mixture with the desired reactant at a chosen temperature in air or under an inert atmosphere of nitrogen or argon gas. Varying the reaction conditions, including for example, catalyst, temperature, solvent, and reagent, to optimize the yield of the desired product would be well within the abilities of a person skilled in the art.

In one embodiment, the disclosure includes the use of compounds of Formula (III) as pharmaceutical products.

In another embodiment, the disclosure includes the use of compounds of Formula (III) for any applications.

### (IV) Benzyl Tetrahydrocannabinols

The present disclosure also includes the preparation of benzyl tetrahydrocannabinols having the following structure:

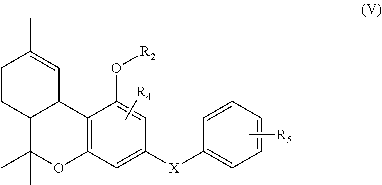

wherein
R₂ is as defined above in any paragraph for compounds of the Formula (I) to Formula (III);
R₄ and R₅ are one or more substitutents which are hydrogen, halo, —OR^{c}, —NR^{c}₂, carboxylates (—COOR, where R is H or (C₁-C₆)-alkyl), phosphates, sulfates, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein R^{c} and R^{d} are independently or simultaneously hydrogen, (C₁-C₂₀)-alkyl, (C₂-C₂₀)-alkenyl, or (C₂-C₂₀)-alkynyl;
X is (C₁-C₁₀-alkylene) or (C₂-C₁₀-alkenylene);
and all isomers, and salts thereof.

In one embodiment, R₄ and R₅ are one or more substitutents which are hydrogen, halo, a (C₁-C₁₀)-alkyl group, or a (C₆-C₁₀)-aryl group. In one embodiment, R₄ and R₅ are one or more substituents which are hydrogen, halo, a (C₁-C₆)-alkyl group, or a phenyl group.

In one embodiment, X is (C₁-C₆-alkylene) or (C₂-C₆-alkenylene). In another embodiment, X is (C₁-C₂-alkylene) or (C₂-alkenylene).

In one embodiment, the compound of the Formula (V) is one of the compounds below:

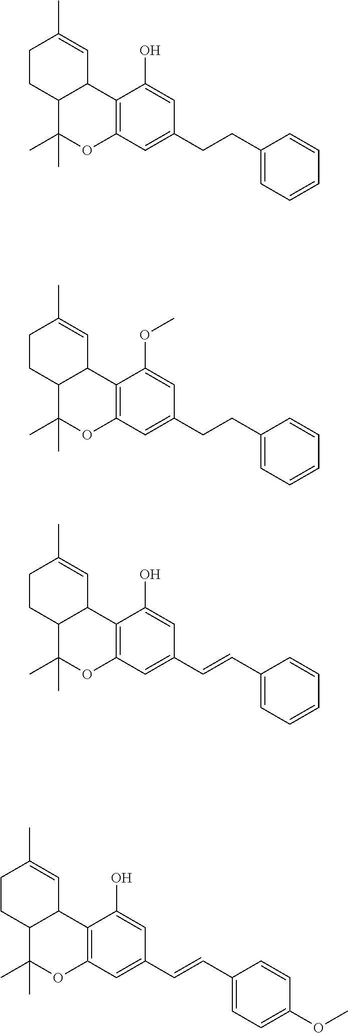 [SMILES: CC1=CC2c3c(O)cc(CCc4ccccc4)cc3OC(C)(C)C2CC1 ; COc1cc(CCc2ccccc2)cc2c1C1C=C(C)CCC1C(C)(C)O2 ; CC1=CC2c3c(O)cc(/C=C/c4ccccc4)cc3OC(C)(C)C2CC1 ; COc1ccc(/C=C/c2cc(O)c3c(c2)OC(C)(C)C2CCC(C)=CC32)cc1]

In some other aspects, the disclosure comprises the preparation of all isomers of compounds of Formula (V). In some other embodiments it comprises the preparation of a mixture of isomers of compounds of Formula (V). In yet some other embodiment it comprises the preparation of single isomers of compounds of Formula (V).

The disclosure also includes the use of compounds of Formula (V), prepared according to the processes of the present disclosure, as pharmaceutical products.

### EXAMPLES

The disclosure will now be described in further details by way of the following examples, wherein the temperatures are indicated in degrees centigrade and the abbreviations have the usual meaning in the art.

All the procedures described hereafter have been carried out under an inert atmosphere unless stated otherwise. All preparations and manipulations under air-free conditions were carried out under N₂ or Ar atmospheres with the use of standard Schlenk, vacuum line and glove box techniques in dry, oxygen-free solvents. Deuterated solvents were degassed and dried over activated molecular sieves. NMR spectra were recorded on a 400 MHz spectrometer (400 MHz for ¹H, 100 MHz for ¹³C, 376 MHz for ¹⁹F and 162 MHz for ³¹P). All ³¹P chemical shifts were measured relative to 85% H₃PO₄ as an external reference. ¹H and ¹³C chemical shifts were measured relative to partially deuterated solvent peaks but are reported relative to tetramethylsilane.

### Example 1. Preparation of (6aR,10aR)-1-hydroxy-6,6,9-trimethyl-6a,7,8,10a-tetrahydro-6H-benzo[c]chromen-3-yl trifluoromethanesulfonate

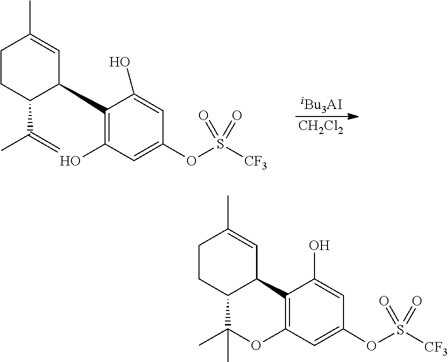

A solution of triisobutylaluminum (2.2 ml of a 1.0 M solution in hexanes, 2.2 mmol) was added to a solution of 3,5-dihydroxy-4-((1R,6R)-3-methyl-6-(prop-1-en-2-yl)cyclohex-2-enyl)phenyl trifluoromethanesulfonate (10.0 g, 25.5 mmol) in dichloromethane (200 ml) and the mixture was stirred at room temperature for 18 hours. The reaction was quenched with ammonium chloride solution and diethyl ether was added. The phases were separated, and the organic layer was dried (MgSO₄), filtered and evaporated to dryness to give the product as a pale-yellow viscous oil. Yield=9.92 g.

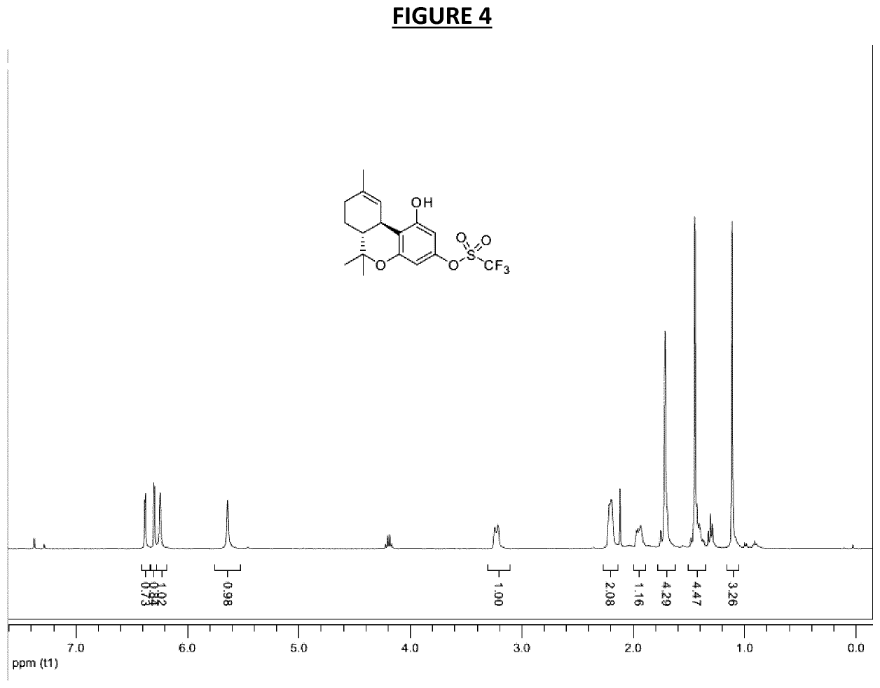

FIG. 4 shows the ¹H NMR spectrum of (6aR,10aR)-1-hydroxy-6,6,9-trimethyl-6a,7,8,10a-tetrahydro-6H-benzo[c]chromen-3-yl trifluoromethanesulfonate.

### Example 2. Preparation of (6aR,10aR)-6,6,9-trimethyl-1-(trimethylsilyloxy)-6a,7,8,10a-tetrahydro-6H-benzo[c]chromen-3-yl trifluoromethanesulfonate

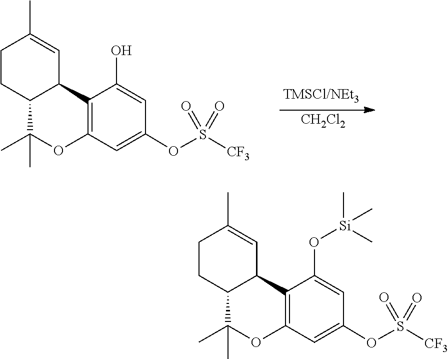

TMSCI (6.36 g, 58.6 mmol) was added to a mixture of (6aR,10aR)-1-hydroxy-6,6,9-trimethyl-6a,7,8,10a-tetrahydro-6H-benzo[c]chromen-3-yl trifluoromethanesulfonate (10.0 g, 25.5 mmol) and NEt₃ (5.92 g, 58.6 mmol) in CH₂Cl₂ (100 ml) at 0° C. The mixture was stirred overnight at room temperature. It was filtered and the solids were washed with dichloromethane. The volatiles were removed from the combined filtrate under reduced pressure. The residue was suspended in hexanes (800 ml) and stirred for 2 hours at room temperature. The mixture was filtered, and the solvent was removed under reduced pressure. The residue was dried under vacuum to give the product as a pale-yellow viscous oil. Yield=11.7 g.

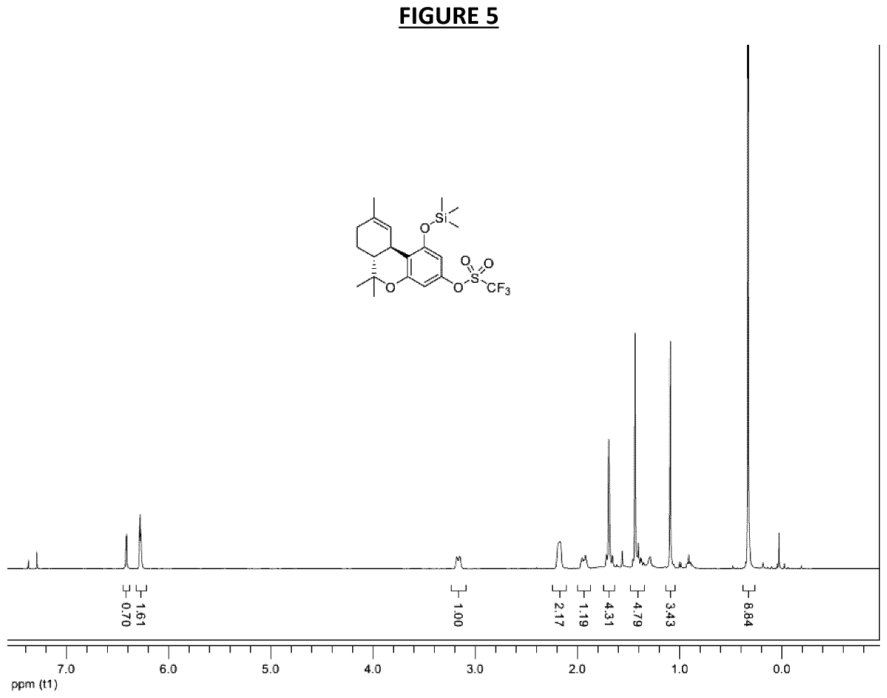

FIG. 5 shows the ¹H NMR spectrum of (6aR,10aR)-6,6,9-trimethyl-1-(trimethylsilyloxy)-6a,7,8,10a-tetrahydro-6H-benzo[c]chromen-3-yl trifluoromethanesulfonate.

### Example 3. (6aR,10aR)-3,6,6,9-tetramethyl-6a,7,8,10a-tetrahydro-6H-benzo[c]chromen-1-ol (THC-C1)

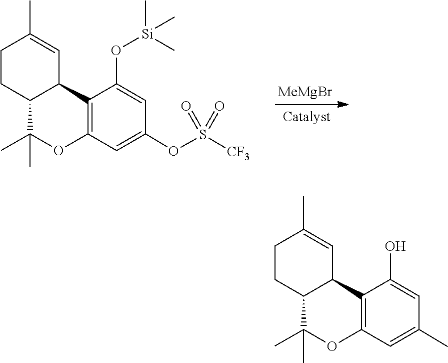

A solution of methylmagnesium bromide (1.4 ml of a 2.0 M solution in diethyl ether, 2.8 mmol) was added to a mixture of ZnBr₂ (0.63 g, 2.8 mmol) in THF (4 ml) and the suspension was stirred for 30 minutes under argon. A solution of (6aR,10aR)-6,6,9-trimethyl-1-(trimethylsilyloxy)-6a,7,8,10a-tetrahydro-6H-benzo[c]chromen-3-yl trifluoromethanesulfonate (1.0 g, 2.15 mmol) in THF (4 ml) and PdCl₂(dppf) (14 mg, 0.019 mmol) were added, and the mixture was stirred at 40° C. for 24 hours under argon. The mixture was cooled to 0° C. and water (2 ml) was added followed by 2M H₂SO₄ (1.0 ml) and the mixture stirred at room temperature for 1 hour. The phases were separated, and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale-yellow oil. Yield=0.54 g.

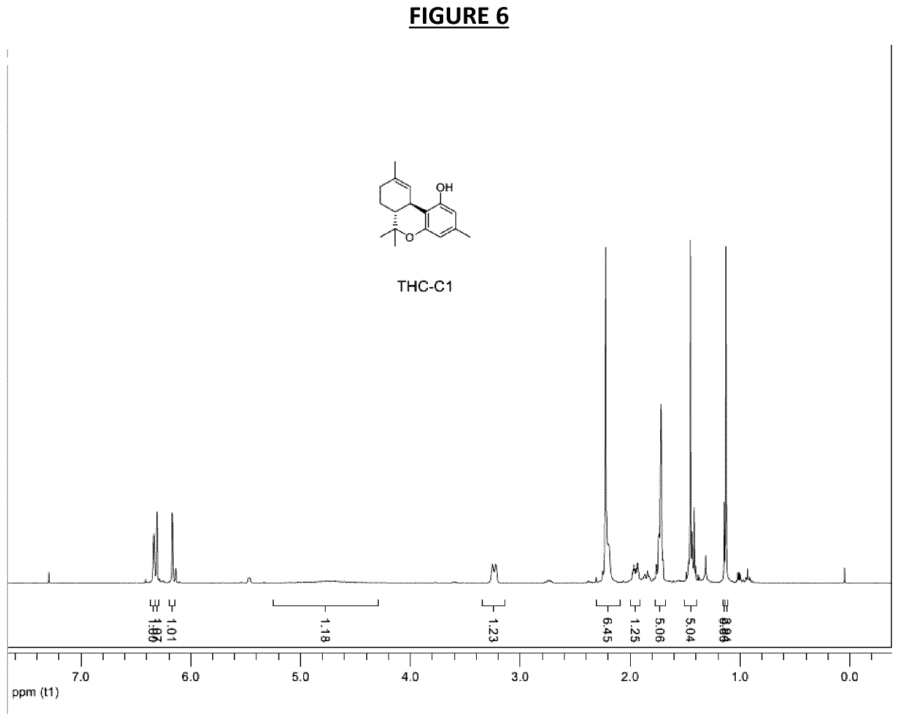

FIG. 6 shows the ¹H NMR spectrum of THC-C1.

### Example 4. (6aR,10aR)-3-ethyl-6,6,9-trimethyl-6a,7,8,10a-tetrahydro-6H-benzo[c]chromen-1-ol (THC-C2)

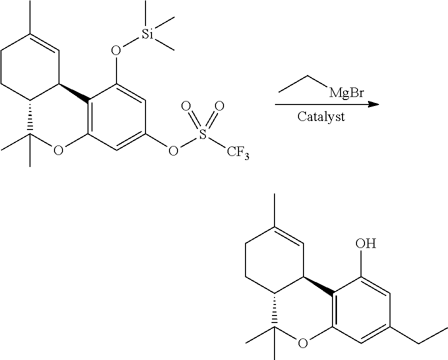

A solution of ethylmagnesium bromide (2.8 ml of a 1.0 M solution in THF, 2.8 mmol) was added to a mixture of ZnBr₂ (0.63 g, 2.8 mmol) in THF (4 ml) and the suspension was stirred for 30 minutes under argon. A solution of (6aR,10aR)-6,6,9-trimethyl-1-(trimethylsilyloxy)-6a,7,8,10a-tetrahydro-6H-benzo[c]chromen-3-yl trifluoromethanesulfonate (1.0 g, 2.15 mmol) in THF (4 ml) and PdCl₂(dppf) (14 mg, 0.019 mmol) were added and the mixture was stirred at 35° C. for 4 hours under argon. The mixture was cooled to 0° C. and water (2 ml) was added followed by 2M H₂SO₄ (1.0 ml) and the mixture stirred at room temperature for 1 hour. The phases were separated, and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale-yellow oil. Yield=0.57 g.

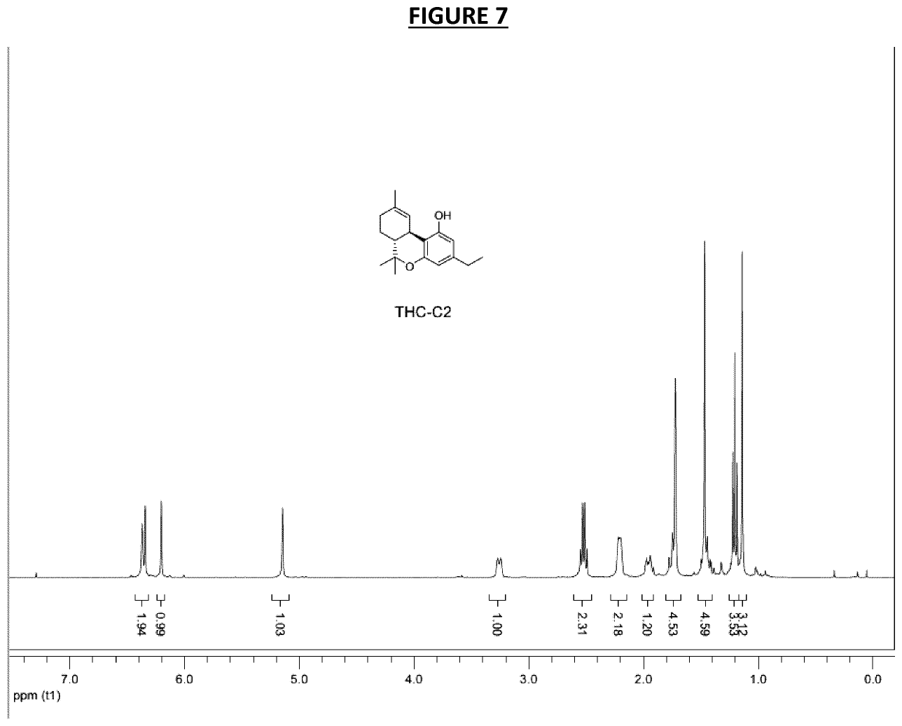

FIG. 7 shows the 1H NMR spectrum of THC-C2.

### Example 5. (6aR,10aR)-6,6,9-trimethyl-3-propyl-6a,7,8,10a-tetrahydro-6H-benzo[c]chromen-1-ol (THCV)

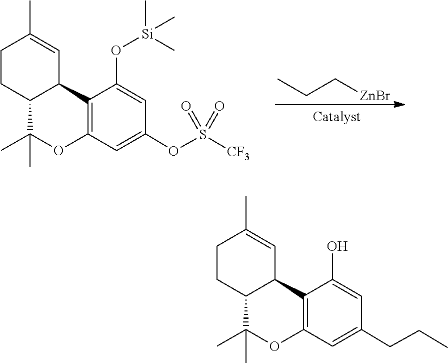

A solution of propylzinc bromide (5.6 ml of a 0.5 M solution in THF, 2.8 mmol) was added to a solution of (6aR,10aR)-6,6,9-trimethyl-1-(trimethylsilyloxy)-6a,7,8,10a-tetrahydro-6H-benzo[c]chromen-3-yl trifluoromethanesulfonate (1.0 g, 2.15 mmol) in THF (4 ml), followed by PdCl₂(dppf) (14 mg, 0.019 mmol) and the mixture stirred at 35° C. for 4 hours under argon. The mixture was cooled to 0° C. and water (2 ml) was added followed by 2M H₂SO₄ (1.0 ml) and the mixture stirred at room temperature for 1 hour. The phases were separated, and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale-yellow oil. Yield=0.60 g.

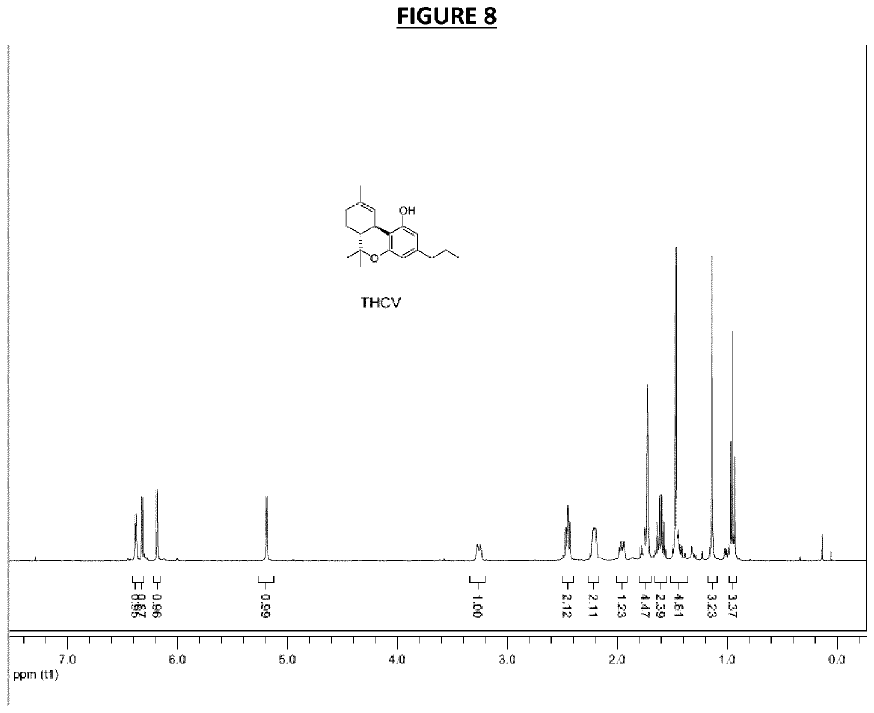

FIG. 8 shows the ¹H NMR spectrum of THCV.

### Example 6. (6aR,10aR)-3-butyl-6,6,9-trimethyl-6a,7,8,10a-tetrahydro-6H-benzo[c]chromen-1-ol (THCB)

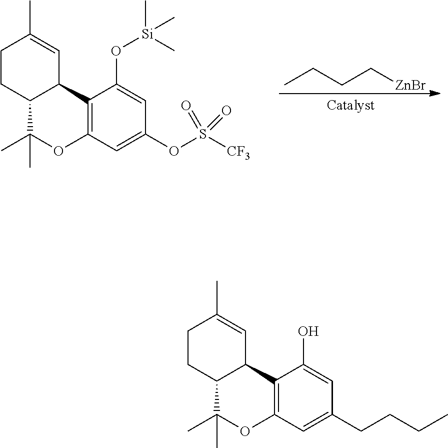

This was prepared using the procedure described in Example 5, using butylzinc bromide.

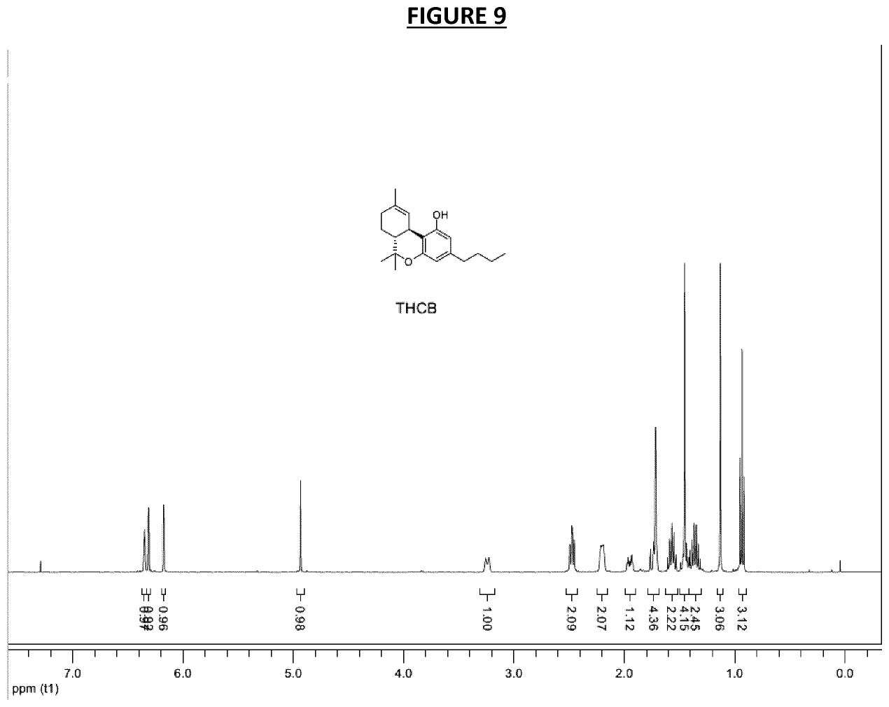

FIG. 9 shows the ¹H NMR spectrum of THCB.

### Example 7. (6aR,10aR)-6,6,9-trimethyl-3-pentyl-6a,7,8,10a-tetrahydro-6H-benzo[c]chromen-1-ol (THC)

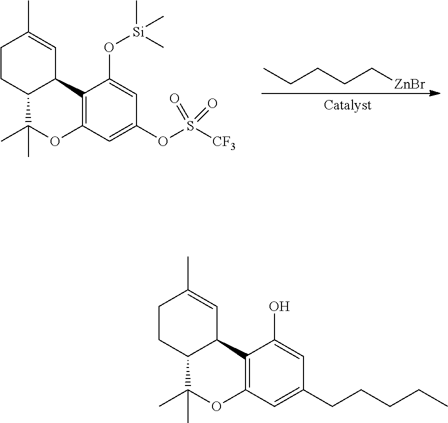

This was prepared using the procedure described in Example 5, using pentylzinc bromide.

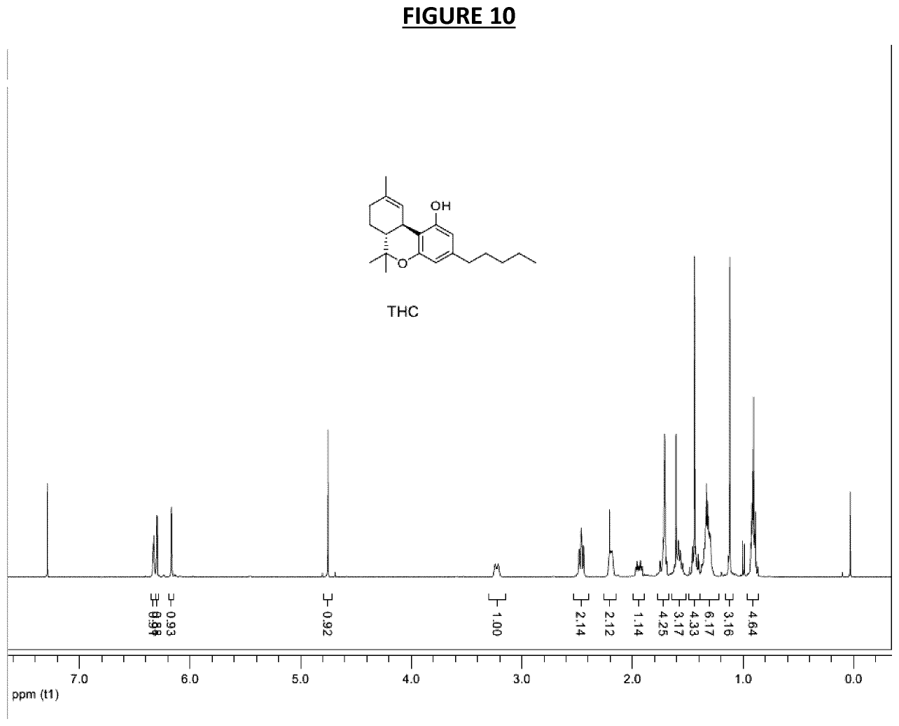

FIG. 10 shows the ¹H NMR spectrum of THC.

### Example 8. (6aR,10aR)-3-hexyl-6,6,9-trimethyl-6a,7,8,10a-tetrahydro-6H-benzo[c]chromen-1-ol (THCH)

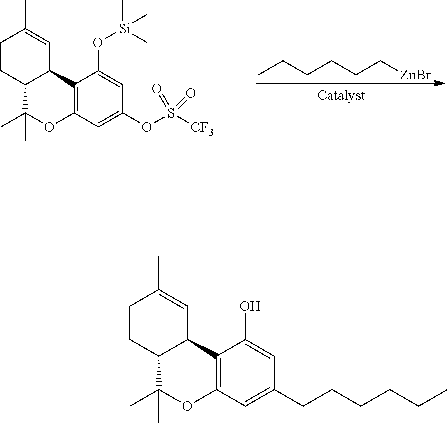

This was prepared using the procedure described in Example 5, using hexylzinc bromide.

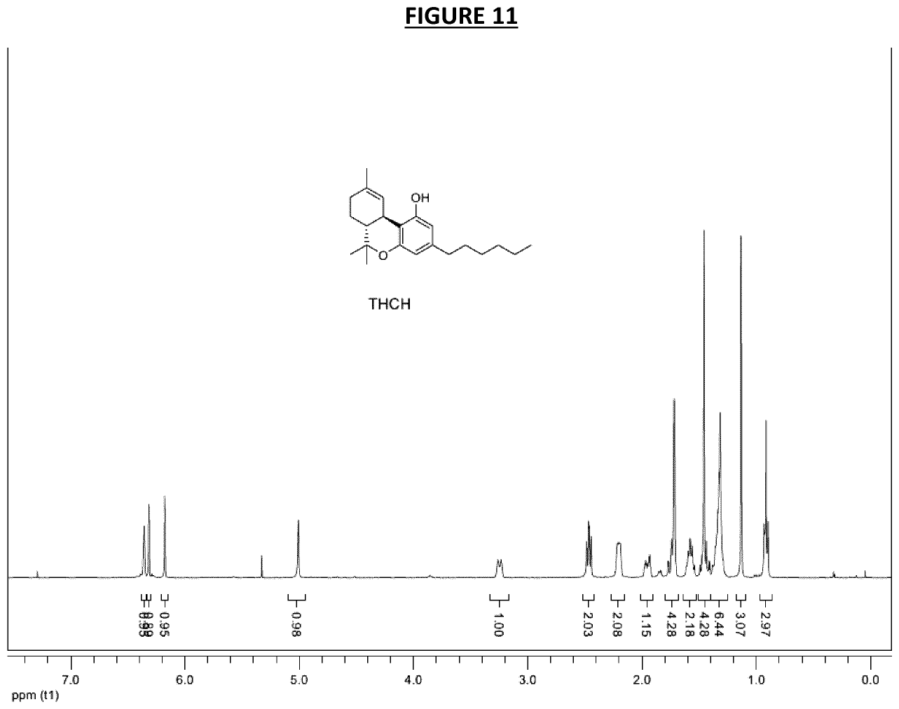

FIG. 11 shows the ¹H NMR spectrum of THCH.

### Example 9. (6aR,10aR)-3-heptyl-6,6,9-trimethyl-6a,7,8,10a-tetrahydro-6H-benzo[c]chromen-1-ol (THCP)

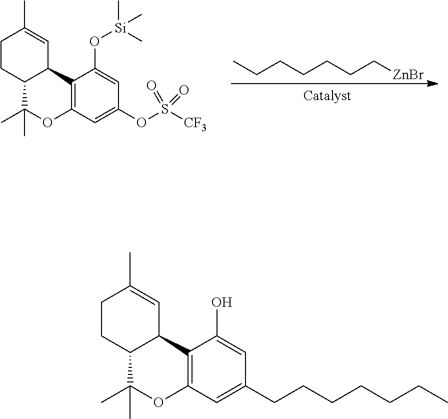

This was prepared using the procedure described in Example 5, using heptylzinc bromide.

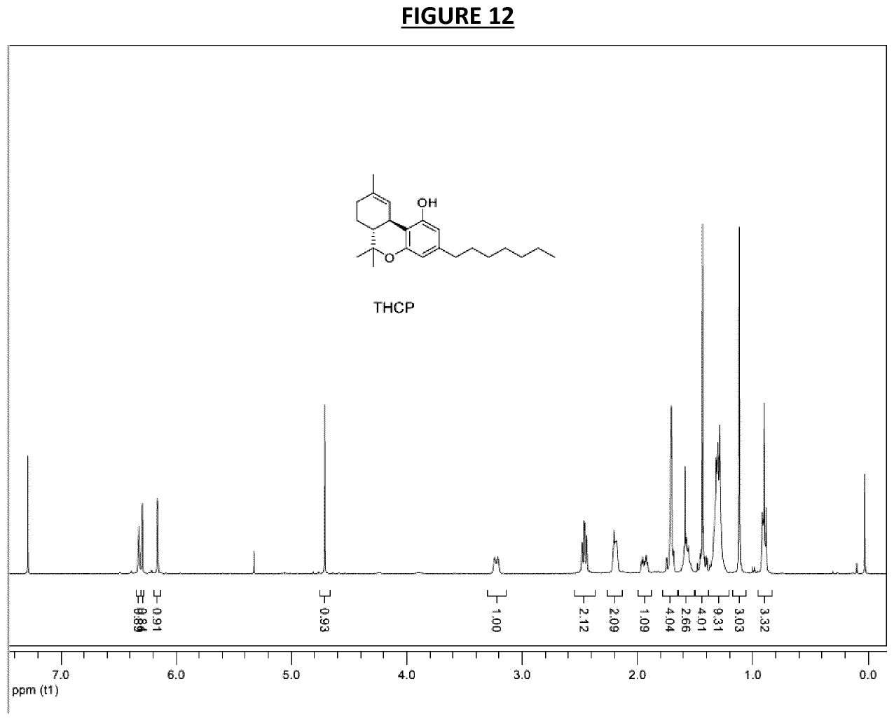

FIG. 12 shows the ¹H NMR spectrum of THCP.

### Example 10. (6aR,10aR)-6,6,9-trimethyl-3-octyl-6a,7,8,10a-tetrahydro-6H-benzo[c]chromen-1-ol (THC-C8)

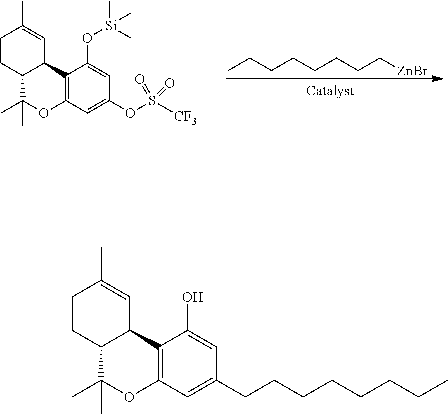

This was prepared using the procedure described in Example 4, using octylmagnesium bromide.

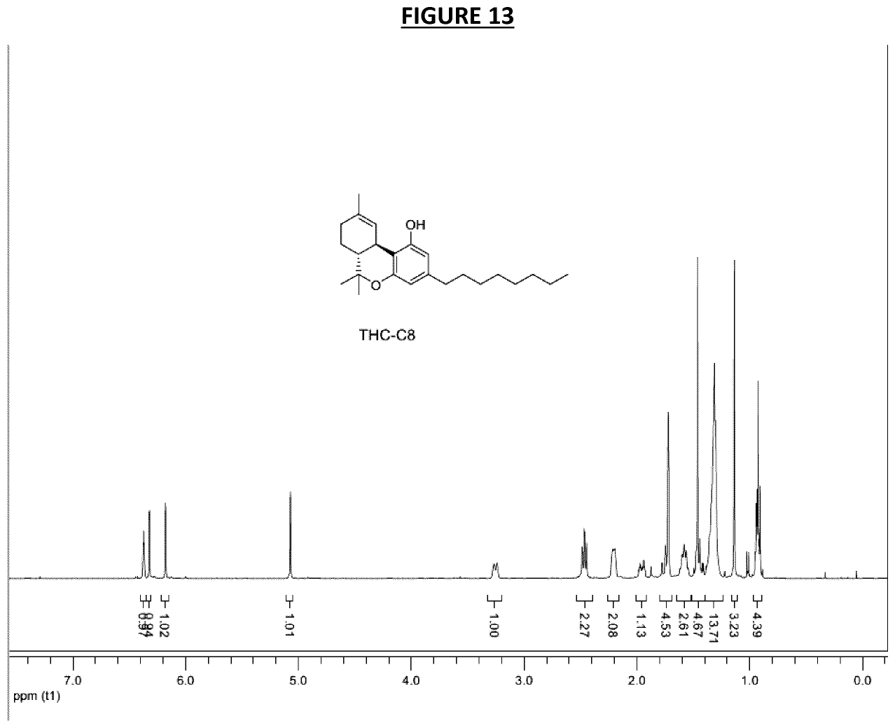

FIG. 13 shows the ¹H NMR spectrum of THC-C8.

### Example 11. (6aR,10aR)-6,6,9-trimethyl-3-nonyl-6a,7,8,10a-tetrahydro-6H-benzo[c]chromen-1-ol (THC-C9)

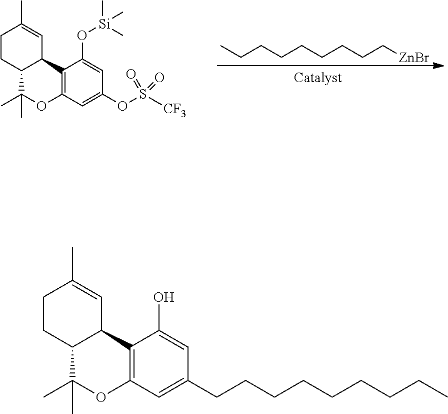

This was prepared using the procedure described in Example 4, using nonylmagnesium bromide.

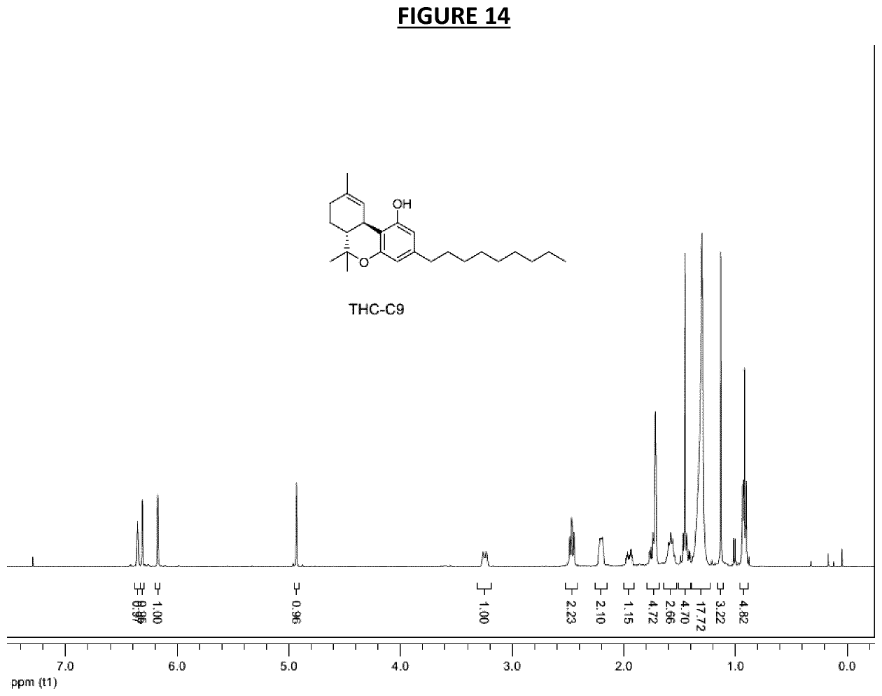

FIG. 14 shows the ¹H NMR spectrum of THC-C9.

### Example 12. (6aR,10aR)-3-decyl-6,6,9-trimethyl-6a,7,8,10a-tetrahydro-6H-benzo[c]chromen-1-ol (THC-C10)

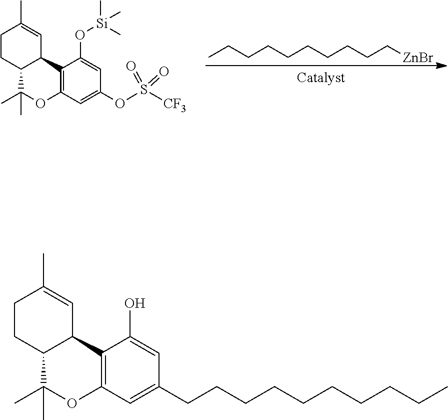

This was prepared using the procedure described in Example 4, using decylmagnesium bromide.

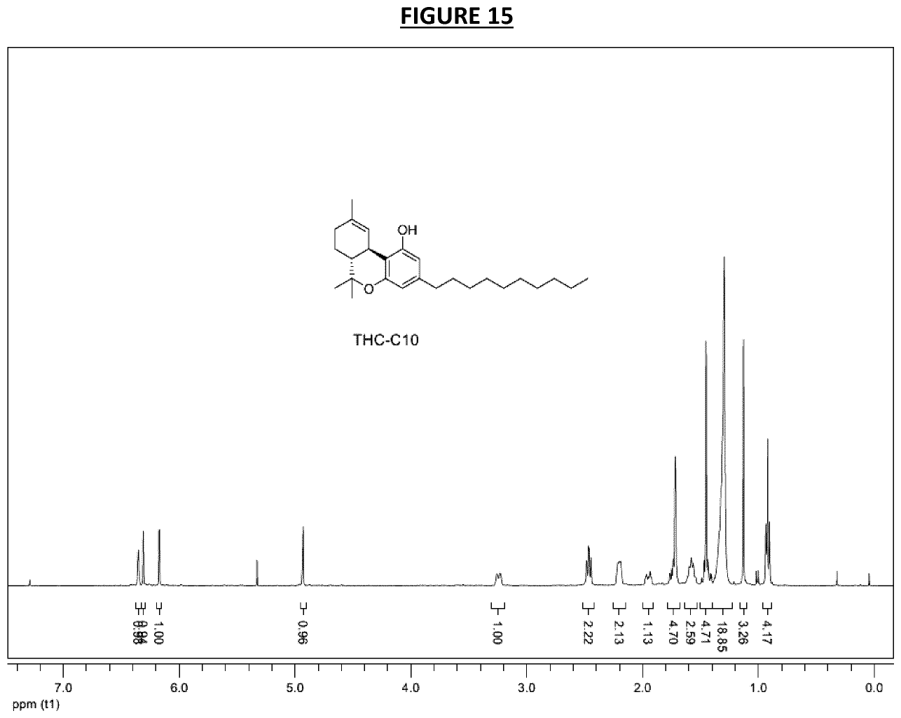

FIG. 15 shows the ¹H NMR spectrum of THC-C10.

### Example 13. (6aR,10aR)-6,6,9-trimethyl-3-phenethyl-6a,7,8,10a-tetrahydro-6H-benzo[c]chromen-1-ol (benzyl-THC)

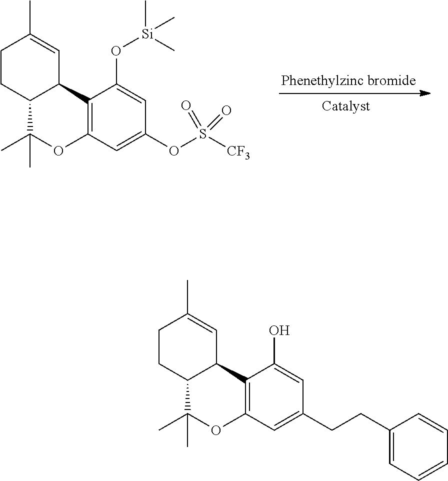 [SMILES: CC1=C[C@H]2c3c(cc(OS(=O)(=O)C(F)(F)F)cc3O[Si](C)(C)C)OC(C)(C)[C@@H]2CC1 ; CC1=C[C@H]2c3c(O)cc(CCc4ccccc4)cc3OC(C)(C)[C@@H]2CC1]

This was prepared using the procedure described in Example 5, using phenethylzinc bromide.

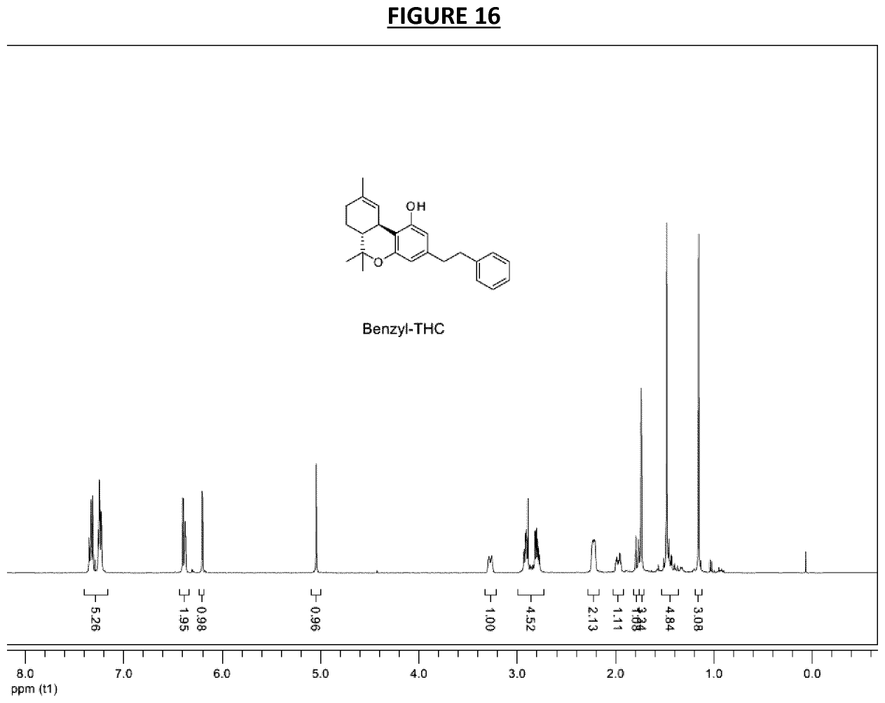

FIG. 16 shows the ¹H NMR spectrum of benzyl-THC.

### Example 14. Preparation of (6aS,10aS)-1-hydroxy-6,6,9-trimethyl-6a,7,8,10a-tetrahydro-6H-benzo[c]chromen-3-yl trifluoromethanesulfonate

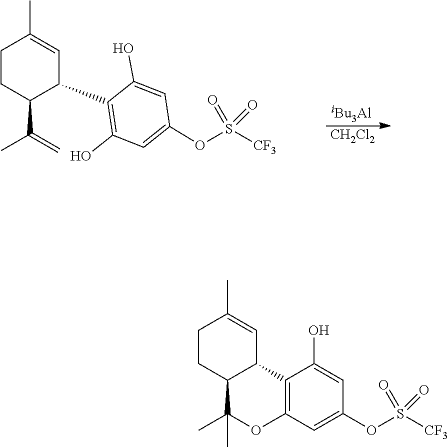 [SMILES: "iBu3Al" ; C=C(C)[C@H]1CCC(C)=C[C@@H]1c1c(O)cc(OS(=O)(=O)C(F)(F)F)cc1O ; "CH2Cl2" ; CC1=C[C@@H]2c3c(O)cc(OS(=O)(=O)C(F)(F)F)cc3OC(C)(C)[C@H]2CC1]

This was prepared using the procedure described in Example 1, using 3,5-dihydroxy-4-((1S,6S)-3-methyl-6-(prop-1-en-2-yl)cyclohex-2-enyl)phenyl trifluoromethanesulfonate.

### Example 15. Preparation of (6aS,10aS)-6,6,9-trimethyl-1-(trimethylsilyloxy)-6a,7,8,10a-tetrahydro-6H-benzo[c]chromen-3-yl trifluoromethanesulfonate

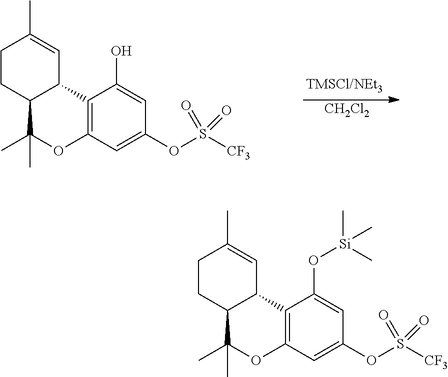 [SMILES: CC1=C[C@@H]2c3c(O)cc(OS(=O)(=O)C(F)(F)F)cc3OC(C)(C)[C@H]2CC1 ; "CH2Cl2" ; CC1=C[C@@H]2c3c(cc(OS(=O)(=O)C(F)(F)F)cc3O[Si](C)(C)C)OC(C)(C)[C@H]2CC1]

This was prepared using the procedure described in Example 2, using (6aS,10aS)-1-hydroxy-6,6,9-trimethyl-6a,7,8,10a-tetrahydro-6H-benzo[c]chromen-3-yl trifluoromethanesulfonate.

### Example 16. (6aS,10aS)-6,6,9-trimethyl-3-propyl-6a,7,8,10a-tetrahydro-6H-benzo[c]chromen-1-ol

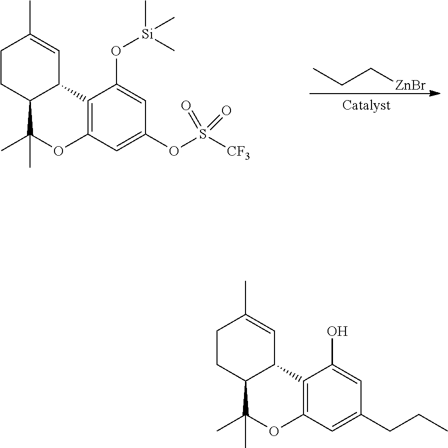

This was prepared using the procedure described in Example 7, using (6aS,10aS)-6,6,9-trimethyl-1-(trimethylsilyloxy)-6a,7,8,10a-tetrahydro-6H-benzo[c]chromen-3-yl trifluoromethanesulfonate and pentylzinc bromide.

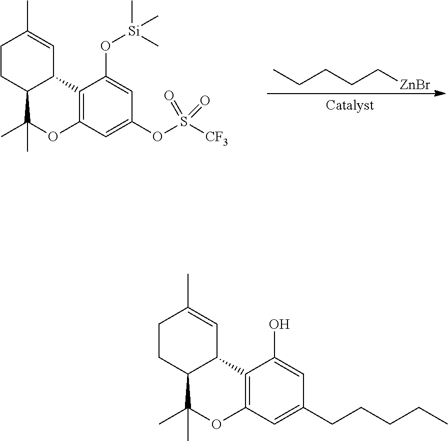

### Example 18. (6aS,10aS)-6,6,9-trimethyl-3-phenethyl-6a,7,8,10a-tetrahydro-6H-benzo[c]chromen-1-ol

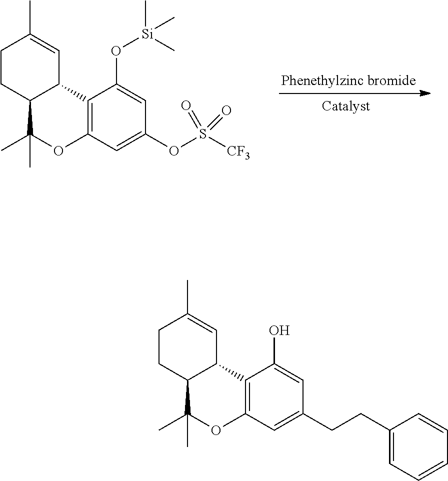 [SMILES: CC1=C[C@@H]2c3c(cc(OS(=O)(=O)C(F)(F)F)cc3O[Si](C)(C)C)OC(C)(C)[C@H]2CC1 ; CC1=C[C@@H]2c3c(O)cc(CCc4ccccc4)cc3OC(C)(C)[C@H]2CC1]

This was prepared using the procedure described in Example 5, using (6aS,10aS)-6,6,9-trimethyl-1-(trimethylsilyloxy)-6a,7,8,10a-tetrahydro-6H-benzo[c]chromen-3-yl trifluoromethanesulfonate and phenethylzinc bromide.

### Example 19. Preparation of (6aS,10aR)-1-hydroxy-6,6,9-trimethyl-6a,7,8,10a-tetrahydro-6H-benzo[c]chromen-3-yl trifluoromethanesulfonate

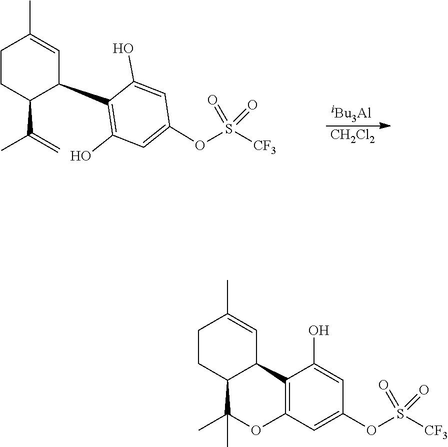 [SMILES: "iBu3Al" ; C=C(C)[C@H]1CCC(C)=C[C@H]1c1c(O)cc(OS(=O)(=O)C(F)(F)F)cc1O ; "CH2Cl2" ; CC1=C[C@H]2c3c(O)cc(OS(=O)(=O)C(F)(F)F)cc3OC(C)(C)[C@H]2CC1]

This was prepared using the procedure described in Example 1, using 3,5-dihydroxy-4-((1R,6S)-3-methyl-6-(prop-1-en-2-yl)cyclohex-2-enyl)phenyl trifluoromethanesulfonate as precursor.

### Example 20. Preparation of (6aS,10aR)-6,6,9-trimethyl-1-(trimethylsilyloxy)-6a,7,8,10a-tetrahydro-6H-benzo[c]chromen-3-yl trifluoromethanesulfonate

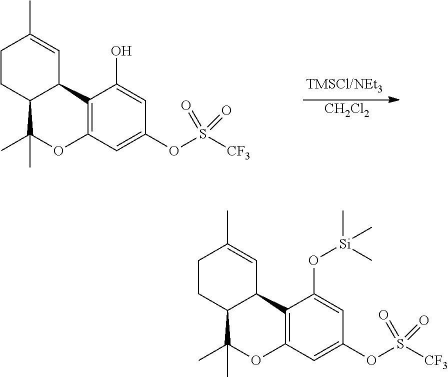 [SMILES: CC1=C[C@H]2c3c(O)cc(OS(=O)(=O)C(F)(F)F)cc3OC(C)(C)[C@H]2CC1 ; "CH2Cl2" ; CC1=C[C@H]2c3c(cc(OS(=O)(=O)C(F)(F)F)cc3O[Si](C)(C)C)OC(C)(C)[C@H]2CC1]

This was prepared using the procedure described in Example 2, using (6aS,10aR)-1-hydroxy-6,6,9-trimethyl-6a,7,8,10a-tetrahydro-6H-benzo[c]chromen-3-yl trifluoromethanesulfonate as precursor.

### Example 21. (6aS,10aR)-6,6,9-trimethyl-3-propyl-6a,7,8,10a-tetrahydro-6H-benzo[c]chromen-1-ol

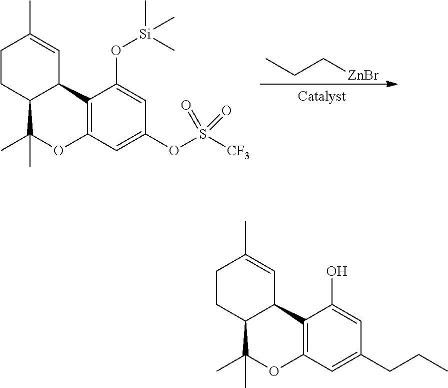

This was prepared using the procedure described in Example 5, using (6aS,10aR)-6,6,9-trimethyl-1-(trimethylsilyloxy)-6a,7,8,10a-tetrahydro-6H-benzo[c]chromen-3-yl trifluoromethanesulfonate and propylzinc bromide.

### Example 22. (6aS,10aR)-6,6,9-trimethyl-3-pentyl-6a,7,8,10a-tetrahydro-6H-benzo[c]chromen-1-ol

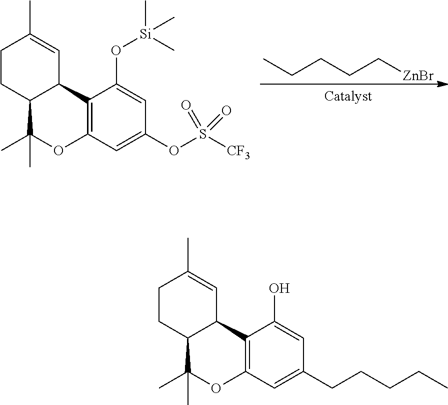

This was prepared using the procedure described in Example 7, using (6aS,10aR)-6,6,9-trimethyl-1-(trimethylsilyloxy)-6a,7,8,10a-tetrahydro-6H-benzo[c]chromen-3-yl trifluoromethanesulfonate and pentylzinc bromide.

### Example 23. (6aS,10aR)-6,6,9-trimethyl-3-phenethyl-6a,7,8,10a-tetrahydro-6H-benzo[c]chromen-1-ol ((−)-Perrottetinene)

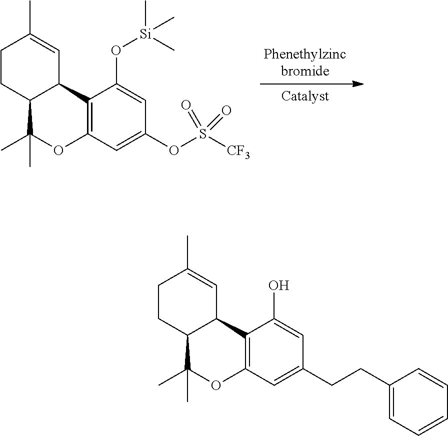 [SMILES: CC1=C[C@H]2c3c(cc(OS(=O)(=O)C(F)(F)F)cc3O[Si](C)(C)C)OC(C)(C)[C@H]2CC1 ; CC1=C[C@H]2c3c(O)cc(CCc4ccccc4)cc3OC(C)(C)[C@H]2CC1]

This was prepared using the procedure described in Example 13, using (6aS,10aR)-6,6,9-trimethyl-1-(trimethylsilyloxy)-6a,7,8,10a-tetrahydro-6H-benzo[c]chromen-3-yl trifluoromethanesulfonate and phenethylzinc bromide.

### Example 24. Preparation of (6aR,10aS)-1-hydroxy-6,6,9-trimethyl-6a,7,8,10a-tetrahydro-6H-benzo[c]chromen-3-yl trifluoromethanesulfonate

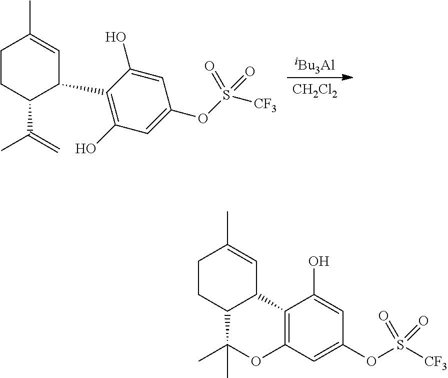

This was prepared using the procedure described in Example 1, using 3,5-dihydroxy-4-((1S,6R)-3-methyl-6-(prop-1-en-2-yl)cyclohex-2-enyl)phenyl trifluoromethanesulfonate as precursor.

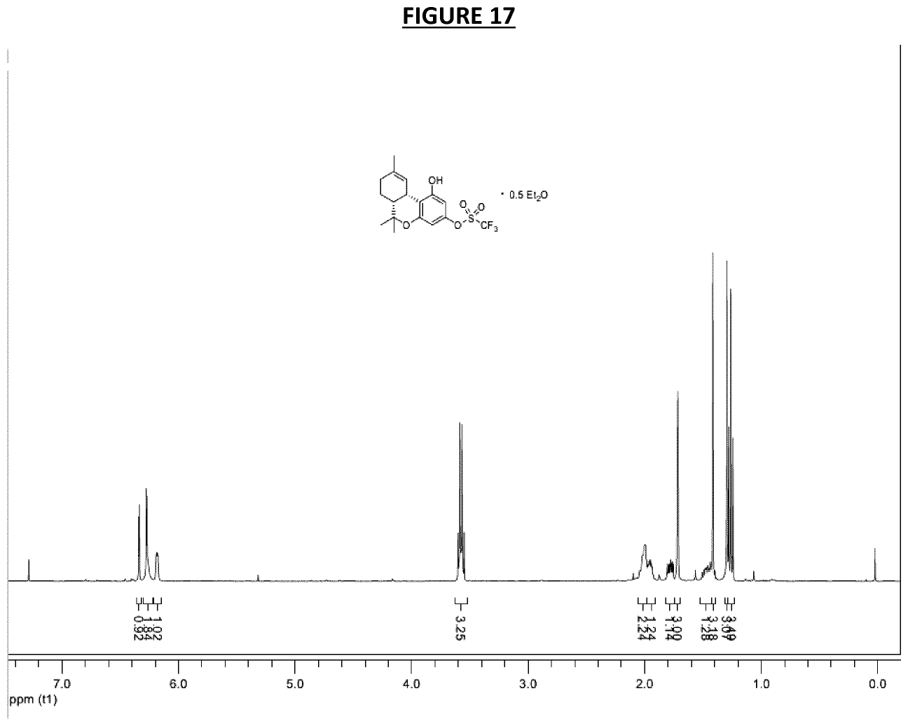

FIG. 17 shows the ¹H NMR spectrum of (6aR,10aS)-1-hydroxy-6,6,9-trimethyl-6a,7,8,10a-tetrahydro-6H-benzo[c]chromen-3-yl trifluoromethanesulfonate/0.5 Et₂O.

### Example 25. Preparation of (6aR,10aS)-6,6,9-trimethyl-1-(trimethylsilyloxy)-6a,7,8,10a-tetrahydro-6H-benzo[c]chromen-3-yl trifluoromethanesulfonate

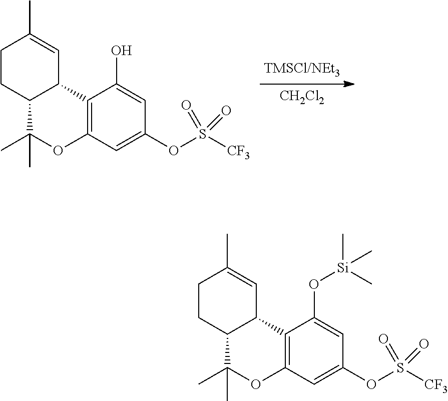

This was prepared using the procedure described in Example 2, using (6aR,10aS)-1-hydroxy-6,6,9-trimethyl-6a,7,8,10a-tetrahydro-6H-benzo[c]chromen-3-yl trifluoromethanesulfonate as precursor.

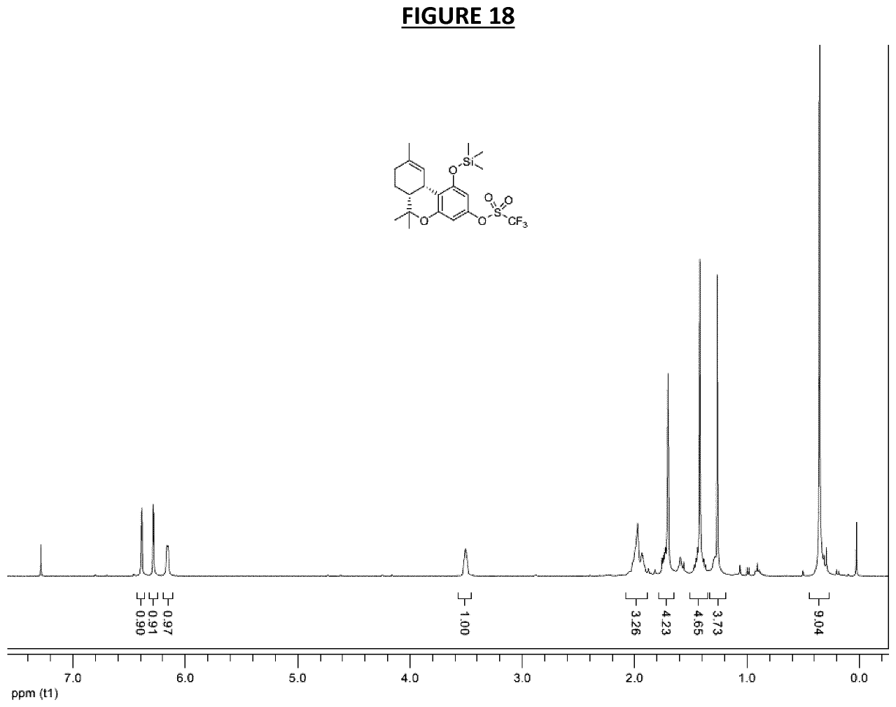

FIG. 18 shows the ¹H NMR spectrum of (6aR,10aS)-6,6,9-trimethyl-1-(trimethylsilyloxy)-6a,7,8,10a-tetrahydro-6H-benzo[c]chromen-3-yl trifluoromethanesulfonate.

### Example 26. (6aR,10aS)-6,6,9-trimethyl-3-propyl-6a,7,8,10a-tetrahydro-6H-benzo[c]chromen-1-ol ((R,S)-THCV)

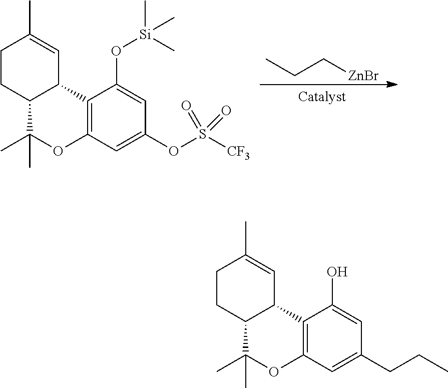

This was prepared using the procedure described in Example 5, using (6aR,10aS)-6,6,9-trimethyl-1-(trimethylsilyloxy)-6a,7,8,10a-tetrahydro-6H-benzo[c]chromen-3-yl trifluoromethanesulfonate and propylzinc bromide.

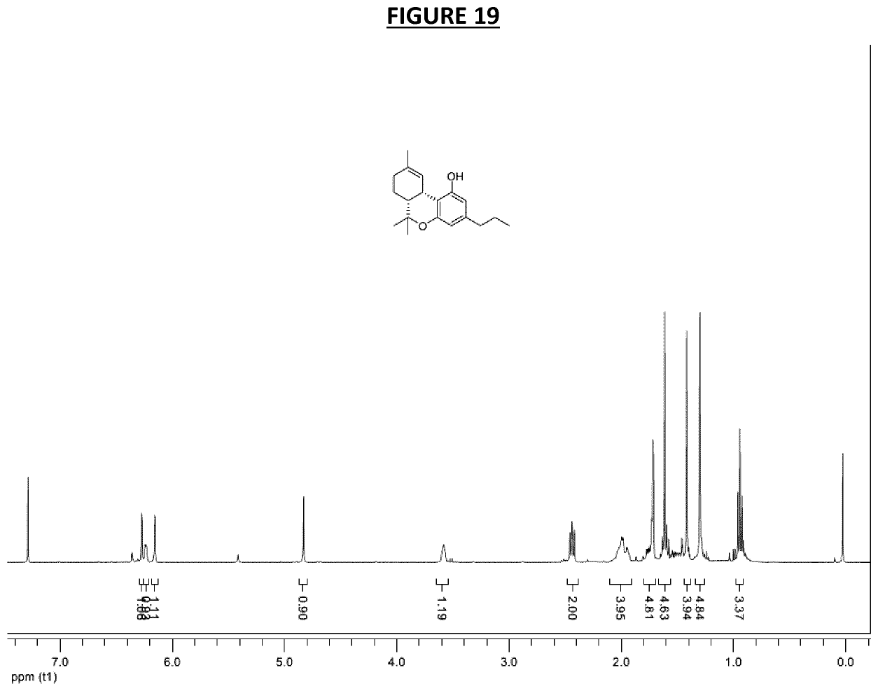

FIG. 19 shows the ¹H NMR spectrum of (R,S)-THCV.

### Example 27. (6aR,10aS)-6,6,9-trimethyl-3-pentyl-6a,7,8,10a-tetrahydro-6H-benzo[c]chromen-1-ol ((R,S)-THC)

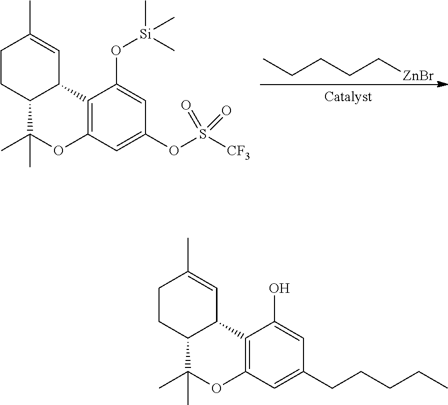

This was prepared using the procedure described in Example 7, using (6aR,10aS)-6,6,9-trimethyl-1-(trimethylsilyloxy)-6a,7,8,10a-tetrahydro-6H-benzo[c]chromen-3-yl trifluoromethanesulfonate and pentylzinc bromide.

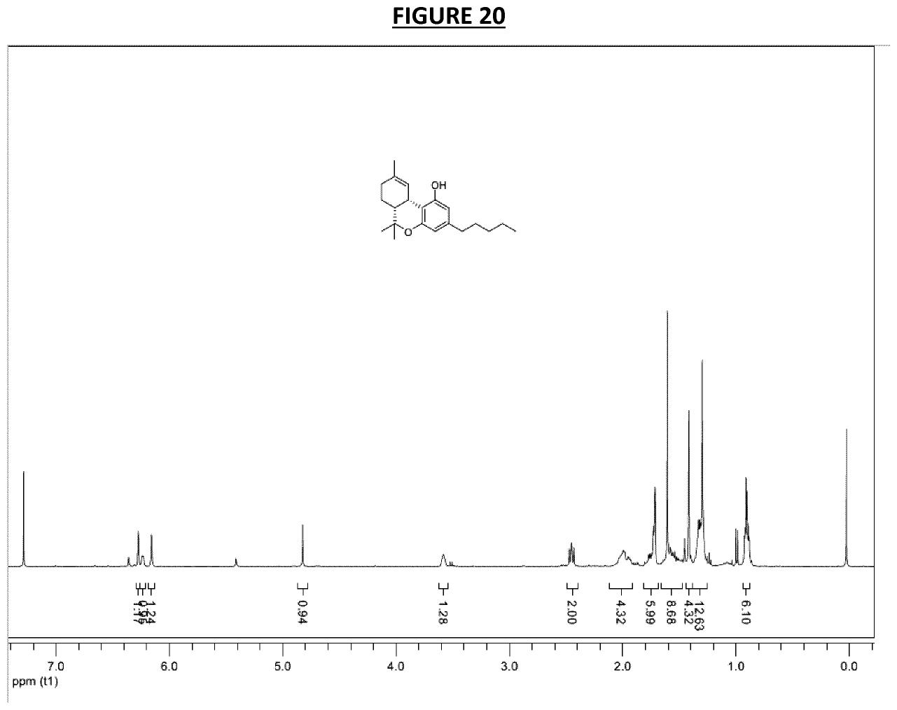

FIG. 20 shows the ¹H NMR spectrum of (R,S)-THC.

### Example 28. (6aR,10aS)-6,6,9-trimethyl-3-phenethyl-6a,7,8,10a-tetrahydro-6H-benzo[c]chromen-1-ol ((+)-Perrottetinene)

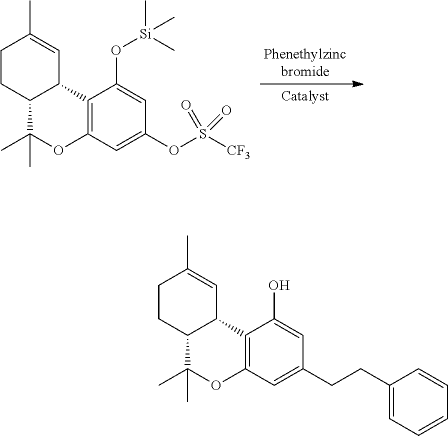 [SMILES: CC1=C[C@@H]2c3c(cc(OS(=O)(=O)C(F)(F)F)cc3O[Si](C)(C)C)OC(C)(C)[C@@H]2CC1 ; CC1=C[C@@H]2c3c(O)cc(CCc4ccccc4)cc3OC(C)(C)[C@@H]2CC1]

This was prepared using the procedure described in Example 13, using (6aR,10aS)-6,6,9-trimethyl-1-(trimethylsilyloxy)-6a,7,8,10a-tetrahydro-6H-benzo[c]chromen-3-yl trifluoromethanesulfonate and phenethylzinc bromide.

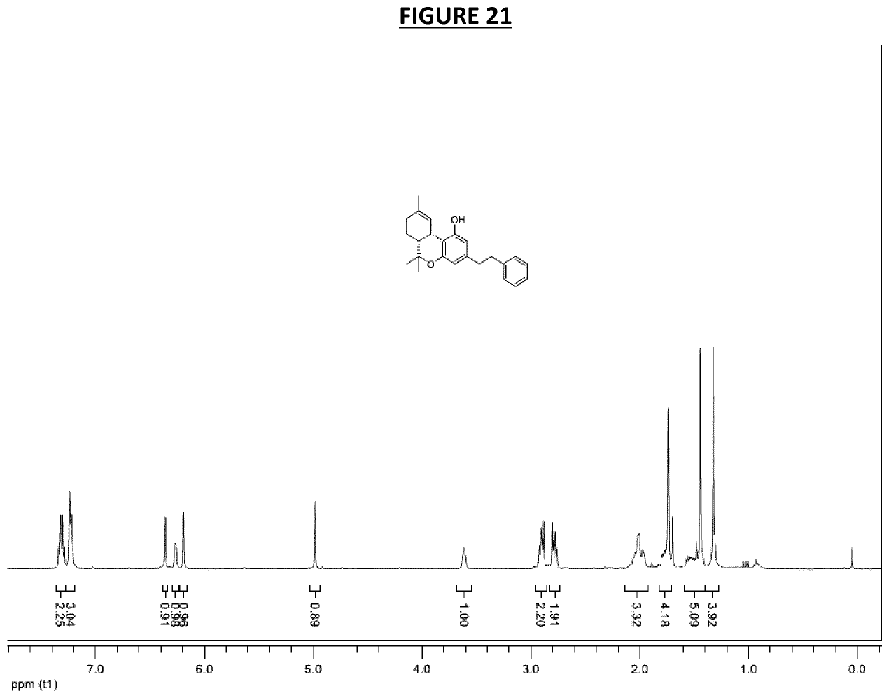

FIG. 21 shows the ¹H NMR spectrum of (+)-Perrottetinene.

### Example 29. Preparation of (6aR,10aR)-6,6,9-trimethyl-3-(trifluoromethylsulfonyloxy)-6a,7,8,10a-tetrahydro-6H-benzo[c]chromen-1-yl benzoate

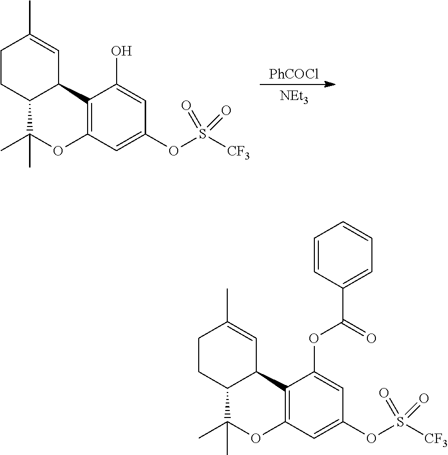 [SMILES: "PhCOCl" ; CC1=C[C@H]2c3c(O)cc(OS(=O)(=O)C(F)(F)F)cc3OC(C)(C)[C@@H]2CC1 ; CC1=C[C@H]2c3c(OC(=O)c4ccccc4)cc(OS(=O)(=O)C(F)(F)F)cc3OC(C)(C)[C@@H]2CC1 ; "NEt3"]

Benzoyl chloride (3.0 g, 21.4 mmol) was added to a mixture of (6aR,10aR)-1-hydroxy-6,6,9-trimethyl-6a,7,8,10a-tetrahydro-6H-benzo[c]chromen-3-yl trifluoromethanesulfonate (8.0 g, 20.4 mmol) and NEt₃ (2.18 g, 21.4 mmol) in ethyl acetate (100 ml) at 0° C. The mixture was stirred at room temperature for 7 hours, then water (100 ml) was added, and the phases separated. The organic layer was washed with NaHCO₃ solution (30 ml), then water (30 ml), then brine, then dried (MgSO₄). It was filtered and the solids were washed with ethyl acetate. The volatiles were removed from the combined filtrate under reduced pressure. Methanol (30 ml) was added to the residue and the resulting suspension was stirred for 2 hours at room temperature. The solids were filtered and washed with methanol, then dried under vacuum to give the product as colourless crystals. Yield=6.15 g.

The mother liquor and washings were combined and evaporated to dryness. Methanol (20 ml) was added, and the suspension stirred for 2 hours. The solids were filtered and washed with methanol, then dried under vacuum to give a second crop of product as colourless crystals. Yield=1.18 g.

Total yield=7.33 g.

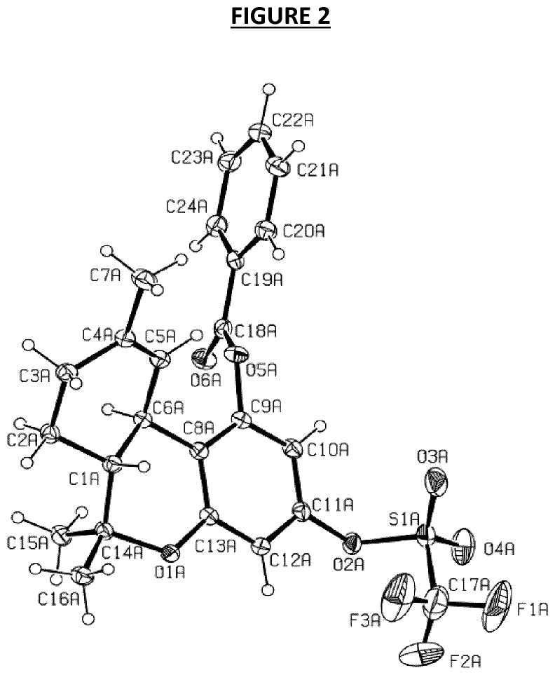

FIG. 2 shows the X-ray crystal structure of (6aR,10aR)-6,6,9-trimethyl-3-(trifluoromethylsulfonyloxy)-6a,7,8,10a-tetrahydro-6H-benzo[c]chromen-1-yl benzoate.

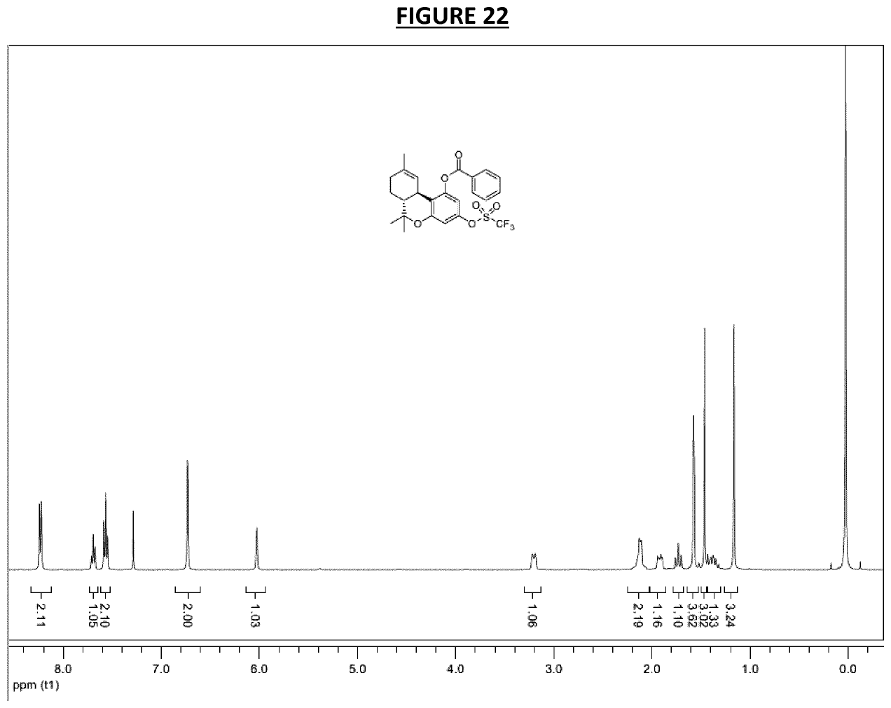

FIG. 22 shows the ¹H NMR spectrum of (6aR,10aR)-6,6,9-trimethyl-3-(trifluoromethylsulfonyloxy)-6a,7,8,10a-tetrahydro-6H-benzo[c]chromen-1-yl benzoate.

### Example 30. Preparation of (6aR,10aR)-6,6,9-trimethyl-3-(trifluoromethylsulfonyloxy)-6a,7,8,10a-tetrahydro-6H-benzo[c]chromen-1-yl 2-naphthoate

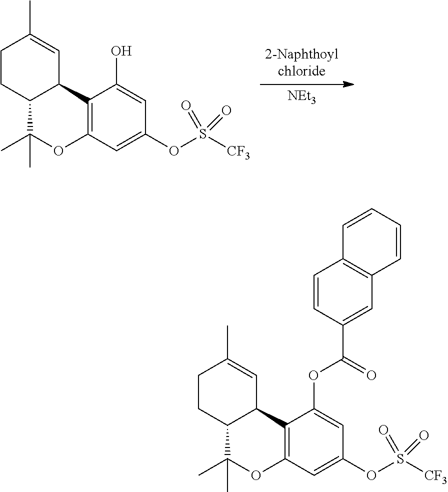 [SMILES: CC1=C[C@H]2c3c(O)cc(OS(=O)(=O)C(F)(F)F)cc3OC(C)(C)[C@@H]2CC1 ; CC1=C[C@H]2c3c(OC(=O)c4ccc5ccccc5c4)cc(OS(=O)(=O)C(F)(F)F)cc3OC(C)(C)[C@@H]2CC1 ; "NEt3"]

2-Naphthoyl chloride (3.09 g, 16.2 mmol) was added to a mixture of (6aR,10aR)-1-hydroxy-6,6,9-trimethyl-6a,7,8,10a-tetrahydro-6H-benzo[c]chromen-3-yl trifluoromethanesulfonate (6.6 g, 16.4 mmol) and NEt₃ (2.75 g, 27.2 mmol) in ethyl acetate (60 ml) and the mixture stirred at room temperature for 8 hours. Water (100 ml) was added, and the phases separated. The organic layer was washed with NaHCO₃ solution (30 ml), then water (30 ml), then brine, then dried (MgSO₄). It was filtered and evaporated to dryness. The residue was suspended in methanol (20 ml) and stirred for 2 hours at room temperature. The solids were filtered and washed with methanol, then dried under vacuum to give the product as colourless crystals. Yield=6.30 g.

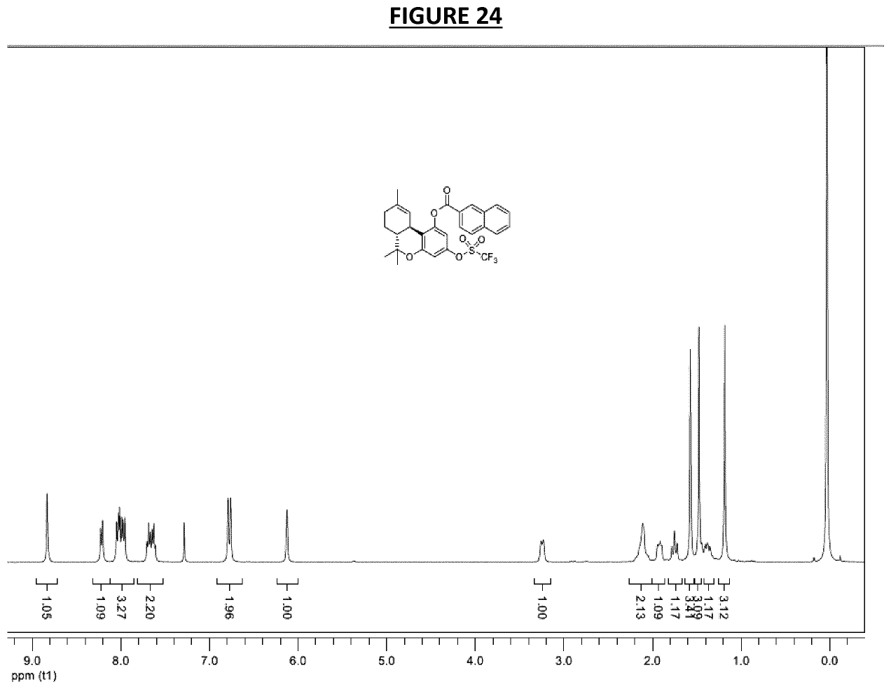

FIG. 24 shows the ¹H NMR spectrum of(6aR,10aR)-6,6,9-trimethyl-3-(trifluoromethylsulfonyloxy)-6a,7,8,10a-tetrahydro-6H-benzo[c]chromen-1-yl 2-naphthoate.

### Example 31. Preparation of (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate

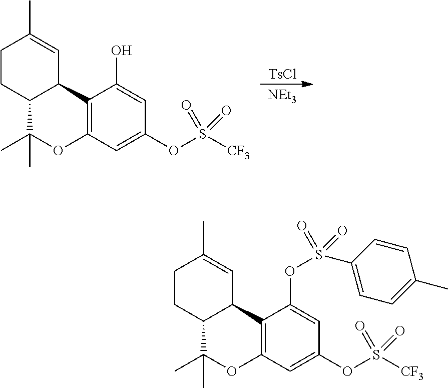 [SMILES: CC1=C[C@H]2c3c(O)cc(OS(=O)(=O)C(F)(F)F)cc3OC(C)(C)[C@@H]2CC1 ; "TsCl" ; CC1=C[C@H]2c3c(cc(OS(=O)(=O)C(F)(F)F)cc3OS(=O)(=O)c3ccc(C)cc3)OC(C)(C)[C@@H]2CC1 ; "NEt3"]

Tosyl chloride (3.0 g, 15.7 mmol) was added to a mixture of (6aR,10aR)-1-hydroxy-6,6,9-trimethyl-6a,7,8,10a-tetrahydro-6H-benzo[c]chromen-3-yl trifluoromethanesulfonate (6.06 g, 15.4 mmol) and NEt₃ (3.1 g, 30.9 mmol) in methylene chloride (80 ml) and the mixture stirred at room temperature overnight. Water (100 ml) was added, and the phases separated. The organic layer was washed with NaHCO₃ solution (30 ml), then water (30 ml), then brine, then dried (MgSO₄). It was filtered and evaporated to dryness. The residue was suspended in cold hexanes (16 ml) and stirred for 2 hours at room temperature. The solids were filtered and washed with cold hexanes, then dried under vacuum to give the product as colourless crystals. Yield=5.82 g.

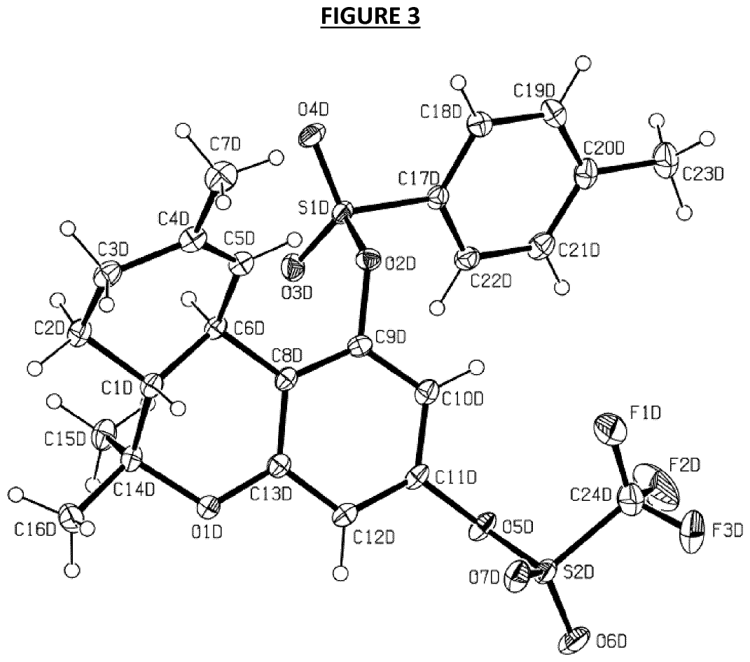

FIG. 3 shows the X-ray crystal structure of (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate.

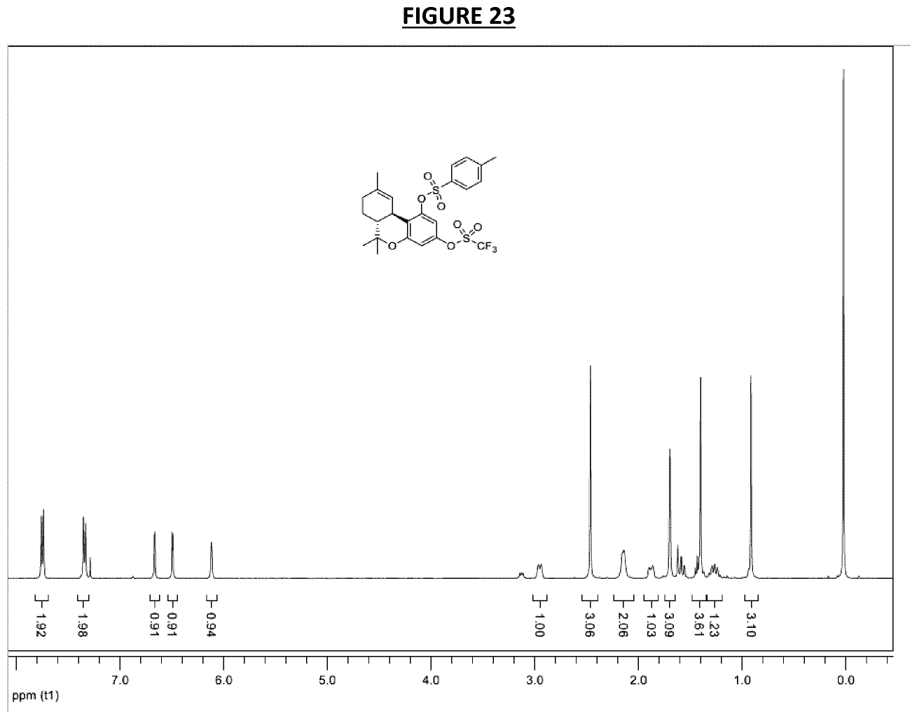

FIG. 23 shows the ¹H NMR spectrum of (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate.

### Example 32. (6aR,10aR)-6,6,9-trimethyl-3-pentyl-6a,7,8,10a-tetrahydro-6H-benzo[c]chromen-1-yl benzoate

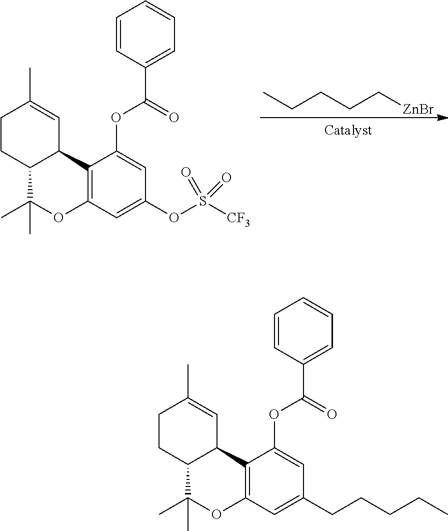

A solution of pentylzinc bromide (5.6 ml of a 0.5 M solution in THF, 2.8 mmol) was added to a solution of (6aR,10aR)-6,6,9-trimethyl-3-(trifluoromethylsulfonyloxy)-6a,7,8,10a-tetrahydro-6H-benzo[c]chromen-1-yl benzoate (1.14 g, 2.3 mmol) in THF (10 ml) at room temperature, followed by PdCl₂(dppf) (17 mg, 0.03 mmol) and the mixture stirred at room temperature for 24 hours under argon. Water (20 ml) along with ethyl acetate (15 ml) were added and the mixture stirred at room temperature for 1 hour. The phases were separated, and the organic layer was washed with brine, then dried (MgSO₄), filtered and evaporated to dryness. The residue was eluted through a short pad of silica gel using diethyl ether/hexanes (1:3) as eluent. The filtrate was evaporated to dryness to give the product as a colourless oil.

Yield=0.90 g.

### Example 33. (6aR,10aR)-6,6,9-trimethyl-3-pentyl-6a,7,8,10a-tetrahydro-6H-benzo[c]chromen-1-yl 2-naphthoate

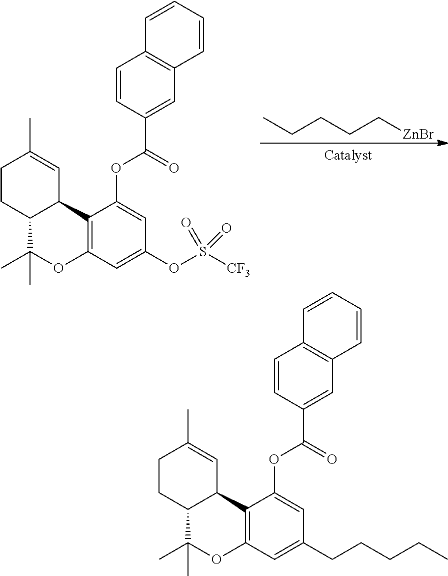

This was prepared using the procedure described in Example 32, using (6aR,10aR)-6,6,9-trimethyl-3-(trifluoromethylsulfonyloxy)-6a,7,8,10a-tetrahydro-6H-benzo[c]chromen-1-yl 2-naphthoate and pentylzinc bromide. The product was isolated as a white crystalline solid. Yield=0.80 g.

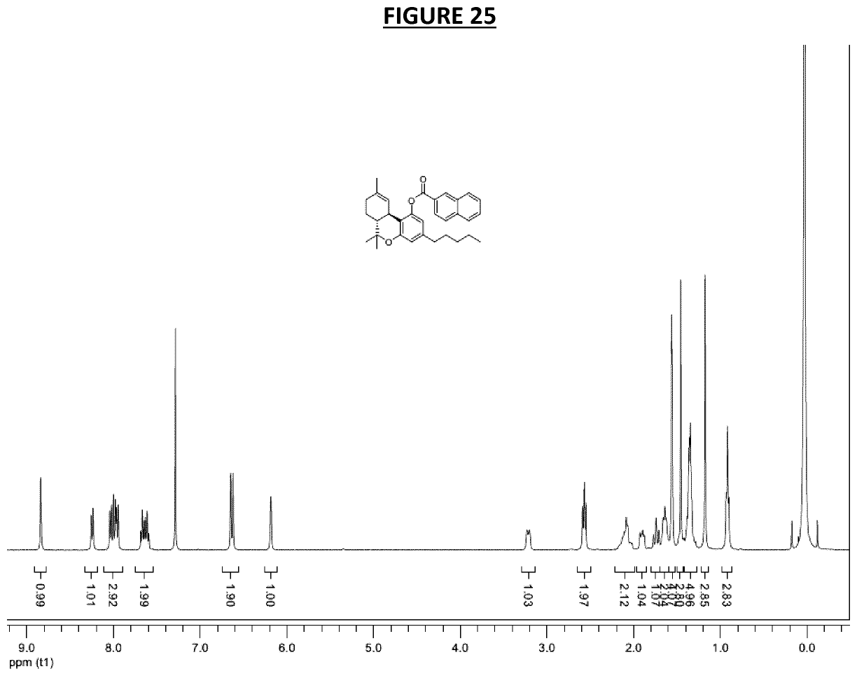

FIG. 25 shows the ¹H NMR spectrum of (6aR,10aR)-6,6,9-trimethyl-3-pentyl-6a,7,8,10a-tetrahydro-6H-benzo[c]chromen-1-yl 2-naphthoate.

### Example 34. (6aR,10aR)-6,6,9-trimethyl-3-pentyl-6a,7,8,10a-tetrahydro-6H-benzo[c]chromen-1-yl 4-methylbenzenesulfonate

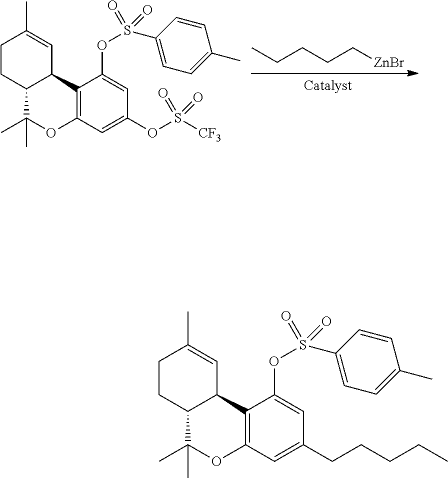

This was prepared using the procedure described in Example 32, using (6aR,10aR)-6,6,9-trimethyl-3-(trifluoromethylsulfonyloxy)-6a,7,8,10a-tetrahydro-6H-benzo[c]chromen-1-yl 4-methylbenzenesulfonate and pentylzinc bromide. The product was isolated as a white crystalline solid. Yield=0.78 g.

### Example 35. Preparation of (6aR,10aR)-6,6,9-trimethyl-3-pentyl-6a,7,8,10a-tetrahydro-6H-benzo[c]chromen-1-ol from (6aR,10aR)-6,6,9-trimethyl-3-pentyl-6a,7,8,10a-tetrahydro-6H-benzo[c]chromen-1-yl 2-naphthoate

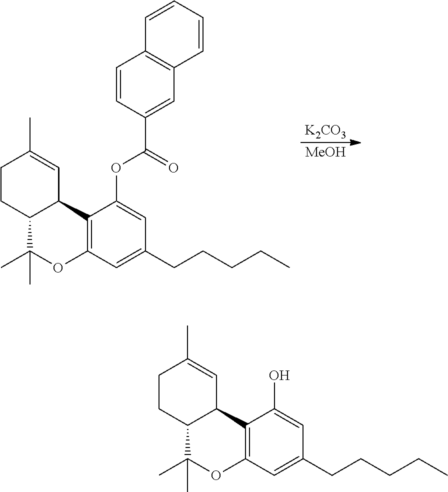 [SMILES: "MeOH" ; CCCCCc1cc(OC(=O)c2ccc3ccccc3c2)c2c(c1)OC(C)(C)[C@@H]1CCC(C)=C[C@@H]21 ; "K2CO3" ; CCCCCc1cc(O)c2c(c1)OC(C)(C)[C@@H]1CCC(C)=C[C@@H]21]

Methanol (5 ml) was added to a mixture of (6aR,10aR)-6,6,9-trimethyl-3-pentyl-6a,7,8,10a-tetrahydro-6H-benzo[c]chromen-1-yl 2-naphthoate (0.50 g, 1.07 mmol) and K₂CO₃ (0.59 g, 4.28 mmol) and the mixture stirred at room temperature for 2 hours under argon. It was filtered and the solvent removed under reduced pressure. Hexanes was added to the residue and the cloudy mixture was eluted through a pad of silica gel using hexanes as eluent. The filtrate was evaporated under vacuum to give the product as a colourless oil. Yield=0.31 g.

While the foregoing disclosure has been described in some detail for purposes of clarity and understanding, it will be appreciated by one skilled in the art, from a reading of the disclosure that various changes in form and detail can be made without departing from the true scope of the disclosure in the appended claims.

All publications, patents, and patent applications are herein incorporated by reference in their entirety to the same extent as if each individual publication, patent or patent application was specifically and individually indicated to be incorporated by reference in its entirety.

1. A compound of Formula (I): 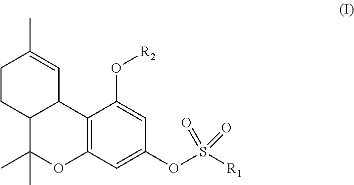
wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;
and R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

2. A compound of Formula (I) according to claim 1, wherein R₂ represents hydrogen.

3. A compound of Formula (I) according to claim 1, wherein R₂ represents a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and
wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

4. (canceled)

5. (canceled)

6. A compound of Formula (I) according to claim 51, wherein R₂ represents a (C₁-C₆)-alkyl group, a —Si[(C₁-C₆)-alkyl]₃ group, or a phenyl group.

7. A compound of Formula (I) according to claim 6, wherein R₂ represents a —Si[(C₁-C₆)-alkyl]₃ group. In one embodiment, R₂ represent a —Si[(C₁-C₃)-alkyl]₃ group.

8. A compound of Formula (I) according to claim 7, wherein R₂ represents a —Si(CH₃)₃ group.

9. (canceled)

10. A compound of Formula (I) according to claim 91, wherein R₁ represents a hydrogen atom, an optionally substituted (C₁-C₁₀)-alkyl group, an optionally substituted (C₂-C₁₀)-alkenyl group, an optionally substituted (C₂-C₁₀)-alkynyl group, an optionally substituted (C₃-C₁₀)-cycloalkyl group, an optionally substituted (C₆-C₁₀)-aryl group, an optionally substituted (C₅-C₁₀)-heteroaryl group, an optionally substituted OR^{c} group or an optionally substituted NR^{c}₂ group.

11. A compound of Formula (I) according to claim 10, wherein R₁ represents a hydrogen atom, an optionally substituted (C₁-C₆)-alkyl group, an optionally substituted (C₂-C₆)-alkenyl group, an optionally substituted (C₂-C₆)-alkynyl group, an optionally substituted (C₃-C₆)-cycloalkyl group, an optionally substituted (Ce)-aryl group, an optionally substituted (C₅-C₆)-heteroaryl group, an optionally substituted OR^{c} group or an optionally substituted NR^{c}₂ group.

12. (canceled)

13. (canceled)

14. A compound of Formula (I) according to claim 1, wherein R₁ represents a hydrogen atom, —CF₃,

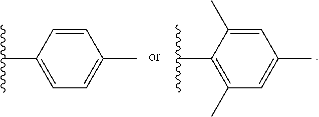

15. A compound of Formula (I) according to claim 1, with one of the structures below:

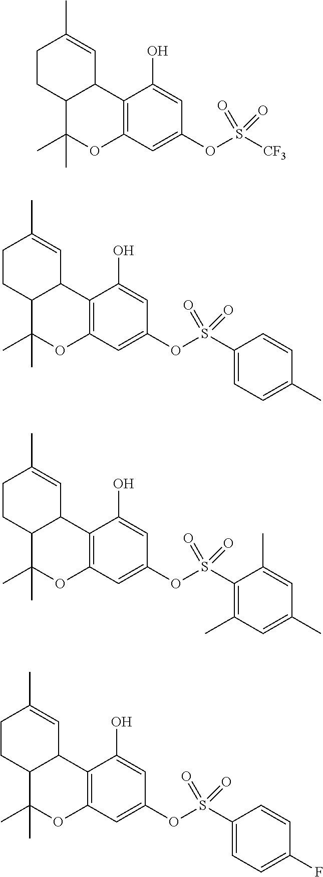 [SMILES: CC1=CC2c3c(O)cc(OS(=O)(=O)C(F)(F)F)cc3OC(C)(C)C2CC1 ; CC1=CC2c3c(O)cc(OS(=O)(=O)c4ccc(C)cc4)cc3OC(C)(C)C2CC1 ; CC1=CC2c3c(O)cc(OS(=O)(=O)c4c(C)cc(C)cc4C)cc3OC(C)(C)C2CC1 ; CC1=CC2c3c(O)cc(OS(=O)(=O)c4ccc(F)cc4)cc3OC(C)(C)C2CC1]

16.-18. (canceled)

19. A compound of Formula (I) according to claim 1, comprising the isomers shown below:

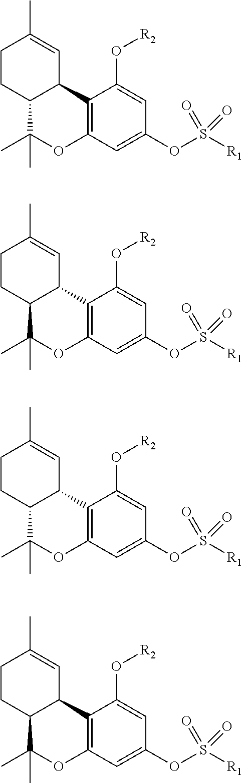

20. (canceled)

21. (canceled)

22. A compound of Formula (II): 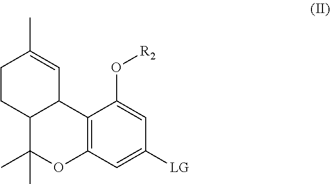
wherein LG is any suitable leaving group, and R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

23. A compound of Formula (II) according to claim 22, wherein LG is:
(i) an anionic group such as sulphonates, halides or boronates;
(ii) MXₙ groups (M=Li, Mg, Zn, Sn, B, Si; X is halide, OH, OR, (C₁-C₂₀)-alkyl, (C₁-C₂₀)-aryl, etc.; n=0 to 3).

24. (canceled)

25. (canceled)

26. (canceled)

27. A compound of Formula (II) according to claim 22, wherein R₂ represents hydrogen.

28.-31. (canceled)

32. A compound of Formula (II) according to claim 22, wherein R₂ represents a —Si[(C₁-C₆)-alkyl]₃ group. In one embodiment, R₂ represent a —Si[(C₁-C₃)-alkyl]₃ group.

33. A compound of Formula (II) according to claim 32, wherein R₂ represents a —Si(CH₃)₃ group.

34.-36. (canceled)

37. A compound of Formula (II) according to claim 22, comprising the isomers shown below:

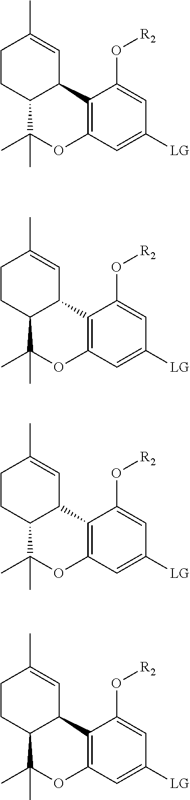

38. (canceled)

39. (canceled)

40. A process for the preparation of compounds of Formula (I) as defined in claim 1, the process comprising contacting a compound of Formula (IV) 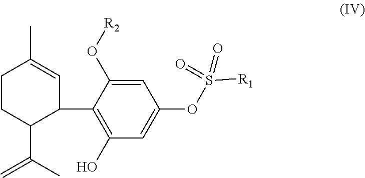
with a catalyst to obtain a compound of Formula (I),
wherein R₁ and R₂ are as defined in any one of claims 1 to 21.

41. (canceled)

42. A process for the preparation of compounds of Formula (III): 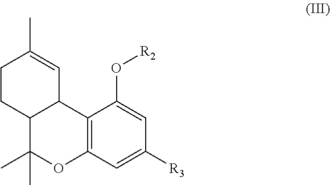
by contacting a compound of Formula (I) as defined in claim 1, or Formula (II) as defined in claim 22, with a nucleophilic R₃ group of the formula R₃—W, wherein
R₃ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl;
W is an electrophilic group, such as a boron containing compound such as R₃—B(OH)₂, R₃—B(OR)₂ or R₃—BF₃K; or a Grignard compound such as R₃—MgX; or an organozinc compound, such as R₃—ZnX, in the presence or absence of a catalyst.

43.-47. (canceled)

48. A process according to claim 42, for the preparation of compounds of Formula (III), wherein R₃ represents a hydrogen atom or a (C₁-C₂₀)-alkyl group optionally substituted with a phenyl group.

49.-54. (canceled)
